SEQUENCE LISTING (2 sequences)

SEQ ID NO: 1 - protein, 451 aa, Artificial Sequence
  heavy chain sequence of the anti-TNFAlpha antibody
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  light chain sequence of the anti-TNFAlpha antibody
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2018/0008707 A1
(43) Pub. Date: Jan. 11, 2018

(54) STABLE LIQUID FORMULATION FOR MONOCLONAL ANTIBODIES

(71) Applicant: SANOFI, Paris (FR)

(72) Inventors: Till BUSSEMER, Frankfurt (DE); Annette PIEPER, Frankfurt (DE); Dijana SAGI, Frankfurt (DE); Nina STREBE, Frankfurt (DE); Ahmed YOUSSEFF, Frankfurt (DE)

(21) Appl. No.: 15/548,598

(22) PCT Filed: Feb. 12, 2016
(86) PCT No.: PCT/EP2016/053068

§ 371 Date: Aug. 3, 2017

(30) Foreign Application Priority Data
Feb. 13, 2015 (EP) 15305218.8

(51) Int. Cl. A61K 39/395; A61K 47/26; A61K 47/22; A61K 47/18; A61K 47/12; A61K 47/02; A61K 9/08; C07K 16/24; A61K 9/00

(52) U.S. Cl. CPC A61K 39/39591; C07K 16/241; A61K 47/26; A61K 47/22; A61K 9/0019; A61K 47/12; A61K 47/02; A61K 9/08; A61K 47/183

(57) ABSTRACT

The present invention concerns stable pharmaceutical compositions for monoclonal antibodies, for example IgG antibodies. The invention further relates to medicaments and treatments using the pharmaceutical compositions of the invention. Additionally, the invention relates to a kit comprising at least one of the pharmaceutical compositions of the invention and a method for reducing aggregation of antibodies.

The present invention concerns stable pharmaceutical compositions for monoclonal antibodies, for example IgG antibodies. The invention further relates to medicaments and treatments using the pharmaceutical compositions of the invention. Additionally, the invention relates to a kit comprising at least one of the pharmaceutical compositions of the invention and a method for reducing aggregation of antibodies.

The instabilities of antibodies are a major obstruction to commercial development of antibody drugs. For instance, certain prior liquid antibody preparations have short shelf lives and antibodies may lose biological activity resulting from chemical and physical instabilities during the storage. Chemical instability may be caused by deamidation, racemization, hydrolysis, oxidation, beta elimination or disulfide exchange, and physical instability may be caused by antibody denaturation, aggregation, precipitation or adsorption. Among those, aggregation, deamidation and oxidation are known to be the most common causes of the antibody degradation (Cleland et al., 1993, Critical Reviews in Therapeutic Drug Carrier Systems 10(4): 307-377). Accordingly, there exists a need for stable compositions of antibodies that bind to antigens of interest, such stable compositions exhibit for instance increased stability, low to undetectable levels of aggregation and low to undetectable levels of antibody fragmentation/degradation. Furthermore, because of the improved physical and chemical stability of said stable compositions, they further have only little to no loss of the biological activities of the antibodies, even during long periods of storage.

The human anti-TNFAlpha monoclonal antibody D2E7, designated as Adalimumab has been developed to treat, for example, rheumatoid arthritis and crohn's disease as described in the international patent application WO199729131. Adalimumab is a monoclonal antibody of the IgG1 class sold by Abbvie in a commercial formulation under the name of Humira®. This antibody is now commonly used in the treatment of plaque psoriasis, crohn's disease, ulcerative colitis, psoriatic arthritis, ankylosing spondylitis, rheumatoid arthritis, polyarticular and juvenile idiopathic arthritis.

The commercial formulation of Humira® has been described in the international patent application WO2004/016286 and in the Annex I of the marketing authorisation granted by the EMA. It contains anti-TNFAlpha antibody, mannitol, citric acid monohydrate, sodium citrate, sodium dihydrogen phosphate dehydrate, sodium chloride, polysorbate 80 and sodium hydroxide.

The inventors discovered that the market formulation of Humira® has a limited stability against mechanical stress.

The inventors developed new compositions comprising a biosimilar of Adalimumab and showed that the compositions of the invention improve the stability of the anti-TNFAlpha antibody to mechanical and thermal stress.

It has been further shown that the compositions of the invention comprising an acetate or histidine buffer, glycine and/or mannitol and a surfactant such as polysorbate minimize the formation of antibody aggregates and particulates. The compositions of the invention exhibit for example a higher content of monomeric antibodies, less aggregates and fragments in comparison to the market formulation (Humira® formulation as described in WO 2004/016286) when the compositions have been stored for example for 1 week at 55° C. (see tables 15 and 23 of the example section).

In particular, size exclusion chromatography (SEC) analysis demonstrated that, after 1 week at 55° C., the tested compositions have a content of antibody monomer that is more than 95% of the total peak area whereas the content of antibody monomer in Humira® formulation is only 89% of the total peak area.

The inventors further demonstrated in long term studies that the compositions comprising at least one acetate or histidine buffer, glycine and/or mannitol and a surfactant such as polysorbate have an amount of monomer that is even after 3 months at 40° C. more than 90% of the total peak area and is thus stable under the conditions tested. Moreover, the inventors showed in stability studies that compositions of the invention have an amount of monomer that represents still more than 99% of the total peak area after exposure to mechanical stress, such as for 3 hours at 200 rpm, and is thus stable under the tested conditions.

Furthermore, the compositions are resistant to stress resulting from freezing and thawing under the tested conditions, accordingly, even in after the 5^{th} cycle the amount of monomer is still more than 99% of the total peak area and is thus stable under the conditions tested.

In another example, the compositions of the invention show a higher mechanical stability with for example regards to particle formation, for instance less amounts of small aggregates in the sub-visible particle size are observed (>1 μm but <10 μm) in comparison to the market formulation (Humira® formulation as described in WO 2004/016286) as shown in example 6, table 24. As further shown in the examples, the effects are especially linked to the choice of buffer (acetate or histidine), the pH range, the addition of mannitol or glycine and the addition of a detergent such as polysorbate. The inventors further showed that asparagine and glutamine have stabilizing effects that are comparable with the stabilizing effect achieved with glycine.

The present invention thus defines suitable pharmaceutical compositions for antibodies such as IgG antibodies, for example anti-hTNFAlpha antibodies, comprising an antibody, an acetate or histidine buffer, at least one amino acid wherein the amino acid is selected from the group consisting of glycine, asparagine and glutamine, for instance glycine and asparagine and/or at least one excipient selected from the group consisting of threhalose and mannitol and a surfactant such as for example polysorbate.

### SUMMARY OF THE INVENTION

The present invention thus relates to a pharmaceutical composition comprising:

a) an antibody,
b) at least one buffer agent selected from the group consisting of acetate and histidine,
c) at least one amino acid selected from the group consisting of glycine, asparagine and glutamine, for example glycine and asparagine, and/or at least one excipient selected from the group consisting of threhalose and mannitol, and
d) a surfactant, wherein the pH of the composition is 5.0 to 6.5.

In one embodiment the at least one excipient is mannitol.

In a further embodiment the at least one amino acid is glycine.

The antibody is a therapeutic antibody. In one embodiment the therapeutic antibody is an IgG antibody. In a further embodiment the IgG antibody is an anti-hTNFAlpha antibody.

The present invention is based on the surprising finding that pharmaceutical compositions having a pH value of 5.0 to 6.5, comprising an acetate or histidine buffer, glycine and/or mannitol and a surfactant such as polysorbate improve the stability of an antibody. Furthermore, the inventors have shown that adding sodium chloride does not favor the stability of anti-hTNF antibodies. Adding sodium chloride has even a negative effect on the denaturating temperature (T_{M}) of the antibody.

Accordingly, in one embodiment, the compositions of the invention comprise less than 10 mg/ml sodium chloride, for example, less than 7 mg/ml sodium chloride. In one embodiment the composition comprises 2 mg/ml sodium chloride or less. In another embodiment the composition does not comprise sodium chloride.

In one embodiment, the surfactant is a polysorbate, for example polysorbate 80 or 20. The composition may comprise 0.01% w/v to 1% w/v surfactant (weight over total volume of the composition).

The compositions of the invention may comprise for example 0.01% w/v of polysorbate 20. In another example the compositions may comprise 0.1% w/v of polysorbate 80.

In one embodiment, the composition may comprise 1 to 100 mM of at least one buffer agent, for example 5 to 50 mM, 5 to 20 mM, for instance 5 to 15 mM, for instance 10 mM.

The compositions of the invention may comprise 30 to 70 mg/ml of antibody, for example 50 mg/ml.

The compositions of the invention may comprise 1 to 30 mg/ml of at least one amino acid.

In one embodiment the at least one amino acid is glycine. The composition may comprise 5 to 30 mg/ml of glycine, for example at 15 mg/ml of glycine.

In another embodiment the at least one amino acid is asparagine. The composition may comprise 1 to 10 mg/ml of asparagine, for example 2 mg/ml of asparagine.

In one embodiment the at least one excipient is trehalose. The composition may comprise 1 to 70 mg/ml of trehalose, for example 50 mg/ml of trehalose.

In another embodiment the at least one excipient is mannitol. The composition may comprise 1 to 60 mg/ml of mannitol, for instance 20 mg/ml of mannitol.

In one embodiment, the invention relates to a pharmaceutical composition comprising:

a) 40 to 50 mg/ml antibody,
b) 5 to 15 mM acetate buffer or histidine buffer,
c) 15 to 25 mg/ml mannitol and/or 10 to 20 mg/ml glycine, and
d) 0.08 to 0.12% w/v polysorbate 80 or 0.008 to 0.012% w/v polysorbate 20,
wherein the pH of the composition is 5.0 to 6.5.

In a further embodiment, the invention relates to a pharmaceutical composition comprising:

a) 50 mg/ml antibody,
b) 5 to 15 mM acetate buffer or histidine buffer,
c) 15 to 25 mg/ml mannitol and/or 10 to 20 mg/ml glycine, and
d) 0.08 to 0.12% w/v polysorbate 80 or 0.008 to 0.012% w/v polysorbate 20,
wherein the pH of the composition is 5.0 to 6.5.

In one embodiment, the invention relates to a pharmaceutical composition comprising:

a) 40 to 50 mg/ml antibody,
b) 5 to 15 mM acetate buffer or histidine buffer,
c) 20 mg/ml mannitol and/or 15 mg/ml glycine, and
d) 0.1% w/v polysorbate 80 or 0.01% w/v polysorbate 20,
wherein the pH of the composition is 5.0 to 6.5.

In a particular embodiment, the invention relates to a pharmaceutical composition comprising

a) 50 mg/ml antibody, and
b) 5 to 15 mM acetate buffer or histidine buffer, and
c) 20 mg/ml mannitol, and/or 15 mg/ml glycine, and
d) 0.1% w/v polysorbate 80 or 0.01% w/v polysorbate 20,
wherein the pH of the composition is 5.0 to 6.5.

The inventors have developed a pharmaceutical composition of anti-TNFAlpha antibodies that that have an improved stability than the same anti-TNFAlpha antibodies when present in the market formulation of Humira®. Therefore, in one embodiment the pharmaceutical composition provides anti-TNFAlpha antibodies having an improved stability in comparison to a reference composition of the same anti-TNFAlpha antibodies.

An improved stability refers for example to an increased physical and/or chemical stability when exposed to stress, wherein the stress may be mechanical stress, thermal stress and/or freeze and thaw stress. The newly developed pharmaceutical compositions of the invention can thus better withstand stress conditions, especially thermal stress and/or mechanical stress than the reference composition.

### DETAILED DESCRIPTION OF THE INVENTION

### Antibody

An “antibody” may be a natural or conventional antibody in which two heavy chains are linked to each other by disulfide bonds and each heavy chain is linked to a light chain by a disulfide bond. There are two types of light chain, lambda (λ) and kappa (κ). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The subclasses IgM, IgD, IgG, IgA and IgE have in the constant region of the heavy chains differences in the amino acid sequences. All immunoglobulins within one given class will have very similar heavy chain constant regions. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site.

As used herein, the term “antibody” denotes conventional antibodies and fragments thereof, as well as single domain antibodies and fragments thereof, for example variable heavy chain of single domain antibodies, and chimeric, humanized, bispecific or multispecific antibodies.

The inventors showed that the pharmaceutical compositions of the invention stabilize anti-hTFNAlpha antibodies in stress conditions such as mechanical stress and/or thermal stress. The anti-hTNFAlpha antibody used in the examples is an IgG antibody. Therefore, in one embodiment the therapeutic antibody is a monoclonal antibody, for instance an IgG antibody.

The term “IgG antibody” covers, among others, the different IgG subclasses (e.g.; IgG1, 2, 3, and 4). The IgG antibody can be divided into subclasses based on small differences in the amino acid sequences in the constant region of the heavy chains. IgG antibodies are molecules of about 150 kDa composed of four peptide chains. It contains two identical class gamma heavy chains of about 50 kDa and two identical light chains of about 25 kDa, thus a tetrameric quaternary structure. The two heavy chains are linked to each other and to one light chain each by disulfide bonds. The resulting tetramer has two identical halves, which together form a fork, or a Y-like shape. Each end of the fork contains an identical antigen binding site.

“Complementarity Determining Regions” or “CDRs” refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

“Framework Regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively.

In one embodiment, the antibody in context of the invention is a therapeutic antibody.

The term “therapeutic antibody” or “therapeutical antibody” or “antibody for therapeutic use” as used herein comprises human, humanized, chimeric and murine antibodies. It further comprises native antibodies isolated from man, mammals, vertrebrates or chordates as well as mutagenized or genetically engineered antibodies.

In one embodiment the antibody refers to antibodies that bind to human TNFAlpha (hTNFAlpha). The antibodies in context of the invention are further characterized by binding to hTNFAlpha but not hTNFBeta (lymphotoxin) and by having the ability to bind to other primate TNFAlphas and non-primate TNFAlphas in addition to human TNFAlpha.

The term “human TNFAlpha” (abbreviated herein as hTNFAlpha, or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of hTNFAlpha is described further in, for example, Pennica, D. et al., 1984 (Nature 312: 724-729), Davis, J. M., et al. 1987 (Biochemistry 26: 1322-1326) and Jones, E. Y., et al., 1989 (Nature 338: 225-228). The mere binding of at least one epitope of TNF alpha inhibits the receptor binding reaction and thus opens a mechanism to treat the below mentioned disorders. In one embodiment, the antibody inhibits or counteracts detrimental hTNFAlpha activity.

In one embodiment the antibody to be used in the frame of the invention may be an anti-TFNAlpha antibody referred to as D2E7 or Adalimumab (Abbvie), or derivatives thereof obtained through the resurfacing technology. Protein sequences of the antibodies mentioned above are publicly available, e.g. referenced in WO2004/016286 (Adalimumab/D2E7) or WO97/29131 (D2E7).

In a further embodiment the pharmaceutical composition as outlined above comprises a therapeutic antibody binding to the targets CXCR5, LAMP 1 or VLA-2.

The term “CXCR5”, as used herein refers to a non-promiscuous receptor. CXCL 13 is a ligand of CXCR5 and is expressed constitutively on stromal cells, such as follicular dendritic cells, and in lymphoid tissues. Moreover, activated T cell induces or upregulate CXCR5 expression. As CXCR5 is selectively expressed on mature B cells, which are linked to the pathogenesis of rheumatoid arthritis, blocking this receptor will modulate the arthritogenic response in affected individuals.

In a particular embodiment, the antibody to be used in the frame of the invention may be an anti-CXCR5 antibody, such as the anti-CXCR5 antibodies disclosed in WO2009/032661 or derivatives thereof obtained through the resurfacing technology. Protein sequences of such antibodies are publicly available, e.g. referenced in WO2009/032661.

As used herein, the term “resurfacing technology” refers to a humanization technology in which non-surface exposed residues of non-human origin are retained, while surface residues are altered to human residues. The resurfacing technology uses a combination of molecular modeling, statistical analysis and mutagenesis to alter the non-CDR surfaces of antibody variable regions to resemble the surfaces of known antibodies of the target host while maintaining the full antigen binding affinity and specificity of the antibody. When the target host is a human, the resurfacing technology thus reduces the immunogenicity of a xenogenic antibody, such as a murine antibody, for introduction into a human. Strategies and methods for the resurfacing of antibodies, and other methods for reducing immunogenicity of antibodies within a different host, are disclosed in U.S. Pat. No. 5,639,641. Briefly, in a preferred method, (1) position alignments of a pool of antibody heavy and light chain variable regions is generated to give a set of heavy and light chain variable region framework surface exposed positions wherein the alignment positions for all variable regions are at least about 98% identical; (2) a set of heavy and light chain variable region framework surface exposed amino acid residues is defined for a rodent antibody (or fragment thereof); (3) a set of heavy and light chain variable region framework surface exposed amino acid residues that is most closely identical to the set of rodent surface exposed amino acid residues is identified; (4) the set of heavy and light chain variable region framework surface exposed amino acid residues defined in step (2) is substituted with the set of heavy and light chain variable region framework surface exposed amino acid residues identified in step (3), except for those amino acid residues that are within 5 Å of any atom of any residue of the complementarity-determining regions of the rodent antibody; and (5) the humanized rodent antibody having binding specificity is produced. Thus in one embodiment “resurfaced” antibodies may also be called humanized antibodies and vice versa.

In one embodiment the antibody to be used in frame of the invention is an anti-TFNAlpha antibody comprising the same heavy and light chain sequence as D2E7 or Adalimumab (Abbvie) or biosimilars thereof.

In a further embodiment the antibody to be used in frame of the invention is a biosimilar of or interchangeable with respect to Adalimumab.

As used herein, “biosimilar” (of an approved reference product/biological drug, such as a protein therapeutic, antibody, etc.) refers to a biologic product that is similar to the reference product based upon data derived from (a) analytical studies that demonstrate that the biological product is highly similar to the reference product notwithstanding minor differences in clinically inactive components; and/or (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biological product. In one embodiment, the biosimilar biological product and reference product utilize the same mechanism or mechanisms of action for the condition or conditions of use prescribed, recommended, or suggested in the proposed labeling, but only to the extent the mechanism or mechanisms of action are known for the reference product. In one embodiment, the condition or conditions of use prescribed, recommended, or suggested in the labeling proposed for the biological product have been previously approved for the reference product. In one embodiment, the route of administration, the dosage form, and/or the strength of the biological product are the same as those of the reference product. In one embodiment, the facility in which the biological product is manufactured, processed, packed, or held meets standards designed to assure that the biological product continues to be safe, pure, and potent. The reference product may be approved in at least one of the U.S., Europe, or Japan.

In one embodiment, the heavy chain of the antibody in context of the invention comprises the heavy chain CDRs (CDR1-H, CDR2-H, CDR3-H) of Adalimumab and the light chain comprises the light chain CDRs (CDR1-L, CDR2-L and CDR3-L) of Adalimumab.

In a further embodiment, the heavy chain of the antibody in context of the invention comprises the heavy chain CDRs (CDR1-H, CDR2-H, CDR3-H) present in the amino acid sequence represented by SEQ ID NO: 1 and the light chain comprises the light chain CDRs (CDR1-L, CDR2-L and CDR3-L) present in the amino acid sequence represented by SEQ ID NO: 2.

In a further embodiment, the heavy chain of the antibody in context of the invention comprises the heavy chain variable domain present in the amino acid sequence represented by SEQ ID NO: 1 and the light chain comprises the light chain variable domain present in the amino acid sequence represented by SEQ ID NO: 2.

In a further embodiment, the antibody in context of the invention comprises a heavy chain consisting of the amino acid sequence represented by SEQ ID NO: 1 and a light chain consisting of the amino acid sequence represented by SEQ ID NO: 2.

CDR/FR definition concerning the immunoglobulin light or heavy chains may be given based on the Kabat definition (http://www.bioinf.org.uk/abs/) or the IMGT definition (Lefranc et al. Dev. Comp. Immunol., 2003, 27(1):55-77; www.imgt.org). Both definitions are known to the skilled in the art and the skilled in the art can thus determine the CDRs and FRs of a given light and heavy amino acid sequence based on those definitions. A mentioned above protein sequences of the antibodies mentioned above are publicly available, e.g. referenced in WO2004/016286 (Adalimumab/D2E7) or WO97/29131 (D2E7).

Further enclosed in the context of the invention are antibodies comprising a sequence with at least 85%, more particularly at least 90%, 91%, 92%, 93%, 94%, 95%, 96%, 97%, 98%, 99% or 100% identical to the sequenced herein disclosed.

A sequence “at least 85% identical to a reference sequence” is a sequence having, on its entire length, 85%, or more, for instance 90%, 91%, 92%, 93%, 94%, 95%, 96%, 97%, 98% or 99% sequence identity with the entire length of the reference sequence.

A percentage of “sequence identity” may be determined by comparing the two sequences, optimally aligned over a comparison window, wherein the portion of the polynucleotide or polypeptide sequence in the comparison window may comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison is conducted by global pairwise alignment, e.g. using the algorithm of Needleman and Wunsch J. Mol. Biol. 48: 443 (1970). The percentage of sequence identity can be readily determined for instance using the program Needle, with the BLOSUM62 matrix, and the following parameters gap-open=10, gap-extend=0.5.

In a specific embodiment, the antibody for use according to the invention is a naked antibody, i.e., it is not linked to any drug in order to form an antibody-drug conjugate.

### Pharmaceutical Compositions

As herein used, the term “pharmaceutical compositions” refers to liquid preparations which are in such form as to permit the biological activity of the active ingredients to be unequivocally effective, and which contain no additional components which are significantly toxic to the subjects to which the composition would be administered. Such compositions are sterile. “Pharmaceutically acceptable” excipients (vehicles, additives) are those which are suitable for administration to a subject.

A “pharmaceutical formulation” or “formulation” refers to the process but also the product of a process in which an active drug is combined with chemical substances to produce a final medicinal product, the final formulation therefore refers to medicinal products such as capsules, pills, tablets, emulsions or compositions. Therefore, in one embodiment, a pharmaceutical formulation is a pharmaceutical composition.

In one embodiment the pharmaceutical composition of the invention is stable.

“Stability” refers to chemical stability and physical stability and can be evaluated qualitatively and/or quantitatively using various analytical techniques that are described in the art and are reviewed in for example Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Those methods include the evaluation of aggregate formation (for example using size exclusion chromatography, by measuring turbidity, and/or by visual inspection); by assessing charge heterogeneity using cation exchange chromatography or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g. Asndeamidation), oxidation (e.g. Met oxidation), isomerization (e.g. Asp isomeriation), clipping/hydro lysis/fragmentation (e.g. hinge region fragmentation), succinimide formation, unpaired cysteine(s), N-terminal extension, C-terminal processing, glycosylation changes, etc. A “deamidated” monoclonal antibody herein is one in which one or more asparagine residue thereof has been modified, e.g. to an aspartic acid or an iso-aspartic acid by a post-translational modification. In order to measure stability a sample of the composition of the invention may be tested in a stability study, wherein a sample is exposed for a selected time period to a stress condition followed by quantitative and qualitative analysis of the chemical and physical stability using an adequate analytical technique.

Accordingly, stability can be measured at a selected temperature for a selected time period for instance by storing a sample at −80° C., −20° C., 5, 25 and 55° C. for up to 1 month and by using for instance SEC, WCX, Light blockage, turbidity and DLS for qualitative and quantitative analysis.

According to the above a “stable composition” is one in which the antibody is physical stable and chemical stable and/or retains its biological activity upon storage.

“Chemical stability” can be assessed by detecting and quantifying chemically altered forms of the antibody. Chemical alteration may involve size modification (e.g. clipping) which can be evaluated for example using size exclusion chromatography, SDS-PAGE and/or matrix-assisted laser desorption ionization/time-of-flight mass spectrometry (MALDI/TOF MS). Other types of chemical alteration include charge alteration (e.g. occurring as a result of deamidation) which can be evaluated for example by ion-exchange chromatography. In context of the invention chemical stability is for example measured by weak cationic exchange chromatography (WCX), wherein a change of 2-3% may be considered as significant.

“Physical stability” refers substantially in context of the invention to an antibody having no signs of aggregation, precipitation and/or denaturation. Methods to access the physical stability are for example size exclusion chromatography (SEC), dynamic light scattering (DLS), light obscuration (LO) and color and clarity. For size exclusion chromatography (SEC) a difference of 1% of the content might be considered as significantly different in the context of the invention under the tested conditions depending on the column used, operating pressure, velocity of the buffer.

An antibody “retains its biological activity” in a pharmaceutical composition, if the antibody in a pharmaceutical composition is biologically active for its intended purpose. For example, biological activity is retained if the biological activity of the antibody in the pharmaceutical composition is within about 30%, about 20%, or about 10% (within the errors of the assay) of the biological activity exhibited at the time the pharmaceutical composition was prepared (e.g., as determined in an antigen binding assay). As known by the skilled in the art of upmost importance for a suitable pharmaceutically active composition is the amount of monomeric antibodies formulated in the solution. Since aggregates may be responsible for causing several as well as severe side effects, the content of monomers displays the actual pharmaceutically active amount of the drug, i.e. the antibody or antibody fragment thereof.

The term “stress” or “stress condition” in context of the invention refers to mechanical stress, thermal stress or stress resulting from freezing and thawing. Methods and conditions to simulate mechanical stress, thermal stress or stress resulting from freezing and thawing are divers and known to the skilled in the art. Mechanical stress may be for example stirring at 200 rpm for 2 to 3 hrs. Thermal stress refers for example to the storage at decreased or increased temperatures for an amount of time, in one example samples may be stored at −80° C., −20° C., 5° C., 25° C. and 40° C., wherein for instance −80° C., −20° C. and 40° C. refer to a stress condition. Samples might be exposed to stress from freezing and thawing by exposing the sample to several cycles of freezing at −80° C. for 24 hrs and thawing at room temperature for 90 min, wherein the cycles are repeated 5 times and wherein the 5^{th} cycle is for example kept longer at −80° C. for 72 hours.

In one embodiment the composition has an increased stability against mechanical stress. Accordingly, the composition may be stressed for example at 200 rpm for at least 2 hours or 3 hrs for example by stirring the samples in vials for instance by using a Variomag Multipoint HP.

In one embodiment the composition has an increased thermal stability. Accordingly, the composition may be stressed at 40° C., 50° C. or 55° C. for at least one week or up to 1 month. The composition may further be stressed at 40° C. for up to 3 or 6 months.

In a further embodiment the composition has an increased stability against stress resulting from freezing and thawing. Accordingly, the composition may be frozen at −80° C. for 24 hrs followed by thawing at room temperature for 90 min, wherein the cycle is for example 5 times repeated. The 5^{th} cycle may be kept for example for 72 hrs at −80° C.

Terms such as “decreased”, “higher”, “less”, “smaller”, “increased”, “lower” or “less” or such alike denote quantitative differences between two states and refer to at least statistically significant differences between the two states.

In one embodiment the composition is stable for 1 week at 55° C.

In a further embodiment, the composition is stable for 1, 3 or 6 months at 40° C.

In a further embodiment the composition is stable after stirring at 200 rpm for 3 hrs.

In an additional embodiment the composition is stable after freezing and thawing, wherein freezing and thawing refers to freezing the composition at −80° C. for 24 hrs followed by thawing at room temperature for 90 min, wherein the cycle is repeated for 5 times repeated and in the 5^{th} cycle the temperature is kept for 72 hrs at −80° C.

In the same embodiments, stable refers to a composition having a monomer % of more than 90%, more than 91%, more than 92%, more than 93%, more than 94%, more than 95%, more than 96%, more than 97% or more than 98% in relation to the total area of all peaks when measured by SEC.

Alternatively, stable may refer to a composition having a having a monomer % content of more than 90%, more than 91%, more than 92%, more than 93%, more than 94%, more than 95%, more than 96%, more than 97%, more than 98% or more than 99%, when analyzed by volume and/or intensity when measured by DLS.

In one embodiment the compositions of the invention have improved stability.

In a further embodiment the compositions of the invention have an improved stability against stress, wherein the stress is selected from mechanical stress, thermal stress or stress resulting from freezing and thawing.

“Improved stability” and/or “increased stability” in context of the invention refer to physical and/or chemical stability that has been qualitatively and/or quantitatively evaluated as described above and which is increased in comparison to the physical and/or chemical stability of a reference composition of the same antibody.

In certain embodiments, the reference composition is the commercially available Adalimumab formulation of WO2004/016286 containing Adalimumab, sodium chloride, monobasic sodium phosphate dihydrate, dibasic sodium phosphate dihydrate, sodium citrate, citric acid monohydrate, mannitol, polysorbate 80, and water for Injection.

In accordance with the above, in one embodiment the pharmaceutical composition of the present invention has at least one feature selected from the group consisting of:

(a) decreased amount of aggregates after storage at about 55° C. for one week as measured by Size Exclusion Chromatography (SEC),
(b) higher amount of monomers after storage at about 55° C. for one week as measured by SEC,
(c) less fragments after storage at about 55° C. for one week as measured by SEC, compared to a reference composition.

Furthermore, in accordance with the above, in one embodiment the pharmaceutical composition of the present invention has at least one feature selected from the group consisting of:

(a) the composition is stable to thermal stress of 1 week at 55° C.,
(b) the composition is stable to mechanical stress of stirring for 3 hours at 55° C., and/or
(c) the composition is stable to stress resulting from freezing and thawing, wherein freezing and thawing refers to freezing the composition at −80° C. for 24 hrs followed by thawing at room temperature for 90 min, wherein the cycle is repeated for 5 times repeated and in the 5^{th} cycle the temperature is kept for 72 hrs at −80° C.,
wherein stability refers to at least one of the following characteristics:
i) the composition has a monomer content in % of more than 90%, more than 91%, more than 92%, more than 93%, more than 94%, more than 95%, more than 96%, more than 97% or more than 98% in relation to the total area of all peaks when measured by SEC,
ii) the composition has an aggregate content in % of less than 3%, less than 2%, less than 1.5%, less than 1.4%, less than 1.3%, less than 1.2%, less than 1.1%, less than 1.0% in relation to the total area of all peaks when measured by SEC, and/or
iii) the composition has a fragment content in % of less than 3%, less than 2%, less than 1.5%, less than 1.4%, less than 1.3%, less than 1.2%, less than 1.1%, less than 1.0% in relation to the total area of all peaks when measured by SEC.

Moreover, in accordance with the above, in one embodiment the pharmaceutical composition of the present invention has at least one feature selected from the group consisting of:

(a) a higher amount of monomers after storage at about 40° C. for three months as measured by Dynamic Light Scattering (DLS), and
(b) a decreased amount of subvisible particles after storage at 40° C. for one to six months as measured by Light Blockage/Light Obscuration (LO),
compared to a reference composition.

Furthermore, in accordance with the above, in one embodiment the pharmaceutical composition of the present invention is stable to thermal stress of 1 to 6 months, in particular of 1, 3 or 6 months, at 40° C., wherein stable refers to at least one of the following characteristics

i) the composition has a monomer content in % of more than 90%, more than 91%, more than 92%, more than 93%, more than 94%, more than 95%, more than 96%, more than 97%, more than 98% or more than 99%, when analyzed by volume measured by DLS,
ii) the composition has a monomer content in % of more than 90%, more than 91%, more than 92%, more than 93%, more than 94%, more than 95%, more than 96%, more than 97%, more than 98% or more than 99%, when analyzed by intensity measured by DLS,
iii) the composition has less than 6000 particles >10 μm, less than 600 particles >25 μm and less than 10000 particles >1 μm when measured by LO.

The inventors have shown that adding sodium chloride does not favor the stability of anti-hTNF antibodies. Furthermore, adding sodium chloride has a negative effect on the denaturating temperature (T_{M}) of the antibody.

Therefore, in one embodiment, the composition comprises less than 7 mg/ml sodium chloride, less than 6 mg/ml, less than 5 mg/ml, less than 2 mg/ml sodium chloride, for example no sodium chloride.

When the composition does not comprise sodium chloride, the composition is essentially free from sodium chlorate.

As used herein the term “essentially” denotes a composition wherein no sodium chlorate molecules are actively, i.e. are intended to be added. Trace amounts of sodium chlorate may be present in a concentration below 5 mg/ml, 3 mg/ml, 2 mg/ml, 1 mg/ml, for example below 0.5 mg/ml, more preferably below 0.05 mg/ml.

In one embodiment the antibody of the composition is comprised in a therapeutically effective amount.

In a pharmacological sense in context of the present invention a “therapeutically effective amount” or “effective amount” of an antibody refers to an amount effective in the prevention or treatment of a disorder for the treatment of which the antibody is effective. Accordingly, the composition may comprise 1 to 150 mg/ml of the antibody, 1 to 140 mg/ml, 10 to 130 mg/ml, 15 to 110 mg/ml, 20 to 100 mg/ml, 25 to 90 mg/ml, 30 to 80 mg/ml, 30 to 70 mg/ml, 40 to 70 mg/ml, 40 to 60 mg/ml of the antibody, for instance 45 to 55 mg/ml, for example 45, 56, 47, 48, 49, 50, 51, 52, 53, 54, 55 mg/ml of the antibody. Ranges intermediate to the above recited concentrations are also intended to be part of this invention. For example, ranges of values using a combination of any of the above recited values as upper and/or lower limits are intended to be included.

As used herein “buffer” refers to a buffered solution that resists changes in pH by the action of its acid-base conjugate components. The “pH” herein refers to the acidity or basicity of the composition at 25° C. Standard methods to measure the pH of a composition are known to the skilled in the art. In one example the pH is typically measured using a pH meter at 25° C. Typically, measuring pH consists of calibrating the instrument, placing the electrodes in a well-mixed sample, and then reading the pH directly from the pH meter. The inventors showed in the screening study that the unfolding temperature of the antibody comprised in the composition is lower at higher buffer concentrations.

The composition thus comprises 1 to 100 mM of at least one buffer agent, 1 to 50 mM of at least one buffer agent, 1 to 30 mM of at least one buffer agent, 1 to 15 mM of at least one buffer agent, for example 5 to 15 mM such as 5 mM, 6 mM, 7 mM, 8 mM, 9 mM, 10 mM, 11 mM, 12 mM, 13 mM, 14 mM, 15 mM of at least one buffer agent. In one embodiment the composition comprises 10 mM of at least one buffer agent.

The inventors showed that acetate and histidine buffers strongly improve stability of the anti-hTFN antibody in comparison to other buffer systems. They further showed that the anti-CXCR5 antibody was stable for up to 6 months at 40° C. in acetate buffer.

Therefore the at least one buffer agent in context of the invention is acetate or histidine.

In one embodiment the at least one buffer agent may be 2, 3 or more buffer agents. Therefore, in one embodiment the at least one buffer agent is two buffer agents. In one example the two buffer agents might be acetate and histidine, wherein the resulting buffer is an acetate-histidine buffer or histidine-acetate buffer.

In one embodiment the at least one buffer is acetate, in the same embodiment the pH of the composition is 5 to 6.5, for example 5.0 to 6.0, for example 5.2 to 5.8, for instance 5.4 to 5.6, for instance 5.4, 5.5, 5.6.

As known by the skilled in the art an acetate buffer consists of a mixture of acetate, for example sodium acetate as the base and acetic acid as the acid. To prepare an acetate buffer of a specific concentration and pH the skilled in the art must calculate the amount of for example sodium acetate or sodium acetate tri-hydrate that has to be mixed with acetic acid. For example for 1 ml of a 10 mM acetate buffer with pH 5.5, 1.17 mg of sodium acetate trihydrate is mixed with 0.08 mg acetic acid, wherein the acetic acid is usually used for pH adjustment. Accordingly, in a particular embodiment, the composition may comprise 1.17 mg/ml sodium acetate trihydrate and 0.08 mg/ml acetic acid, such that the composition comprises 10 mM acetate buffer and is of pH 5.5.

In one embodiment the at least one buffer is histidine, in the same embodiment the pH of the composition is 5 to 6.5, for example 5.5 to 6.5, for example 5.7 to 6.3, for instance 5.9 to 6.1, for instance 5.9, 6.0, 6.1.

Histidine (pK 5.97) is a preferred buffer for subcutaneous, intramuscular and peritoneal injection.

The inventors further tested the stabilizing effect of different excipients, for instance sugars and polyols. They discovered surprisingly, that trehalose and mannitol showed the highest Tₘ temperatures in μDSC experiments.

The compositions of the invention thus comprise in one embodiment at least one excipient selected from the group consisting of trehalose and mannitol, for example mannitol.

In one embodiment of the invention, the composition comprises 1 to 70 mg/ml of excipient, for example 1 to 60 mg/ml of excipient, 10 to 50 mg/ml of the at least one excipient.

Trehalose is a non-reducing sugar which is an alpha-linked disaccharide formed by an alpha, alpha-1,1-glucoside bond between two alpha-glucose units (alpha-D-glucopyranosyl-(1→1)-alpha-D-glucopyranoside). In one embodiment the composition comprises 1 to 70 mg/ml of trehalose, for example 10 to 70 mg/ml of trehalose, for instance 20 to 70 mg/ml of trehalose, for example 20, 25, 30, 35, 40, 45, 50, 55, 60, 65 and 70 mg/ml trehalose, for instance 50 mg/ml trehalose.

In one embodiment the composition may comprise 1 to 60 mg/ml of mannitol, 1 to 50 mg/ml, for example 1 to 40 mg/ml, for example 7.5 to 40 mg/ml of mannitol, for instance 7.5 to 30 mg/ml of mannitol, for instance 15 to 25 mg/ml of mannitol, for example 7.5, 10, 12, 14, 16, 18, 20, 22, 24, 26, 28, 30 mg/ml, for example 12 mg/ml or 20 mg/ml. In one embodiment the at least one excipient may be 2, 3 or more excipients. Therefore, in one embodiment the at least one excipient are mannitol and trehalose.

The inventors further tested the stabilizing effect of amino acids, wherein glycine, L-asparagine, and glutamine showed the highest denaturating temperatures for the antibody in μDSC experiments.

The term “amino acid” as used herein denotes a pharmaceutically acceptable organic molecule possessing an amino moiety located at a-position to a carboxylic group. Examples of amino acids include: arginine, glycine, ornithine, lysine, histidine, glutamic acid, asparagic acid, isoleucine, leucine, alanine, phenylalanine, tyrosine, tryptophane, methionine, serine, and proline. The amino acid employed is optionally in the L-form.

In one embodiment the composition comprises 1 to 50 mg/ml, 1 to 40 mg/ml, for example 1 to 30 mg/ml of the at least one amino acid.

In one embodiment the at least one amino acid is glycine. The composition may comprise 5 to 30 mg/ml of glycine, for example 10 to 20 mg/ml of glycine, for instance 12 to 16 mg/ml of glycine, for example 12, 13, 14, 15, 16, for instance 15 mg/ml.

In a further embodiment, the at least one amino acid may be asparagine. The composition may comprise 1 to 20 mg/ml of Asparagine, for example 1 to 10 mg/ml of Asparagine, such as 1, 2, 3, 4, 5, 6, 7, 8, 9, 10, for instance 2 mg/ml.

The term “surfactant” and “detergent” may be used herein interchangeably. Exemplary detergents include nonionic detergents such as polysorbates (e.g. polysorbates 20, 80 etc) or poloxamers (e.g. poloxamer 188). The amount of detergent added is such that it reduces aggregation of the formulated antibody and/or minimizes the formation of particulates in the composition and/or reduces adsorption.

In one embodiment the surfactant is a polysorbate. A polysorbate is an emulsifier derived from PEG-ylatedsorbitan (a derivative of sorbitol) esterified with fatty acids. This class of agents comprises, among others, polysorbates 20, 21, 40, 60, 61, 65, 80, 81, 85, and 120.

In one further embodiment the composition comprises the surfactant polysorbate 20 (common commercial brand names include Alkest TW 20 and Tween 20) and/or polysorbate 80 (common commercial brand names include Alkest TW 80, Canarcel, Poegasorb 80, Tween 80). The composition may comprise 0.001% w/v to 1% w/v of surfactant, for instance 0.001% w/v to 0.15% w/v of surfactant, for example 0.01% w/v to 0.15% w/v of surfactant.

In one embodiment, the composition comprises 0.001% w/v to 0.15% w/v of polysorbate 80, for example 0.01% w/v to 0.15% w/v, for example 0.05% w/v to 0.15% w/v, for example 0.08% w/v to 0.12% w/v for example 0.08, 0.085, 0.09, 0.095, 0.1, 0.115, 0.12% w/v of polysorbate 80. The composition of the invention comprises for instance about 0.1% w/v polysorbate 80.

In another embodiment, the composition comprises 0.001% w/v to 0.15% w/v of polysorbate 20, for example 0.005% w/v to 0.1% w/v, for example 0.008% w/v to 0.05% w/v, for example 0.008% w/v to 0.015% w/v, for example 0.008% w/v to 0.012% w/v, for example 0.008, 0.009, 0.01, 0.011, 0.012, 0.013, 0.014, 0.015% w/v. The composition of the invention comprises for instance 0.01% w/v polysorbate 20.

According to one example the composition of the invention comprises 50 mg/ml antibody, 10 mM acetate buffer at pH 5.5, 20 mg/ml mannitol, 15 mg/ml glycine and 0.1% w/v polysorbate 80 (PS80). More particularly, according to one example the composition of the invention comprises 50 mg/ml antibody, 1.17 mg/ml sodium acetate trihydrate, 0.08 mg/ml acetic acid, 20 mg/ml mannitol, 15 mg/ml glycine and 0.1% w/v PS80, the pH of the composition being 5.5.

According to another example the composition of the invention comprises 50 mg/ml antibody, 10 mM acetate buffer at pH 5.5, 20 mg/ml mannitol, 15 mg/ml glycine and 0.01% w/v polysorbate 20 (PS20). More particularly, according to another example the composition of the invention comprises 50 mg/ml antibody, 1.17 mg/ml sodium acetate trihydrate, 0.08 mg/ml acetic acid, 20 mg/ml mannitol, 15 mg/ml glycine and 0.01% w/v PS20, the pH of the composition being 5.5.

According to a further example, the composition of the invention comprises 41 mg/ml antibody, 10 mM acetate buffer at pH 5.5, 20 mg/ml mannitol, 15 mg/ml glycine and 0.1% w/v PS80.

According to a further example the composition of the invention comprises 50 mg/ml antibody, 10 mM acetate buffer at pH 5.5, 20 mg/ml mannitol, 15 mg/ml glycine, 0.1% w/v PS80 and 2 mg/ml NaCl.

According to a further example the composition of the invention comprises 50 mg/ml antibody, 10 mM acetate buffer at pH 5.5, 15 mg/ml glycine and 0.01% w/v PS20.

According to a further example the composition of the invention comprises 50 mg/ml antibody, 10 mM acetate buffer at pH 5.5, 12 mg/ml mannitol, 0.1% w/v PS80 and 6.165 mg/ml NaCl.

According to a further example the composition of the invention comprises 50 mg/ml antibody 7.45 mM histidine buffer at pH 6.0, 12 mg/ml mannitol, 0.1% w/v PS80 and 6.165 mg/ml NaCl.

In one embodiment one or more other pharmaceutically acceptable carriers, excipients or stabilizers such as those described in Remington's Pharmaceutical Sciences 16th edition, Osol, A. Ed. (1980) may be included in the composition provided that they do not significantly adversely affect the desired characteristics of the composition. Acceptable carriers, excipients or stabilizers are nontoxic to recipients at the dosages and concentrations employed and include additional buffering agents; co-solvents; antioxidants including ascorbic acid and methionine; chelating agents such as EDTA; metal complexes (e.g. Zn-protein complexes); biodegradable polymers such as polyesters; and/or salt-forming counterions such as sodium.

The composition of the invention may also be combined with one or more other therapeutic agents as necessary for the particular indication being treated, preferably those with complementary activities that do not adversely affect the antibody of the composition. Such therapeutic agents are suitably present in combination in amounts that are effective for the purpose intended.

### Medicaments and Treatments Using the Pharmaceutical Composition

In one embodiment the invention provides a method of treating or preventing a disease or disorder comprising administering to a subject in need thereof a therapeutically effective amount of a pharmaceutical composition of the invention.

The invention also relates to a pharmaceutical composition of the invention for use as a medicament. The invention further refers to the use of a pharmaceutical composition of the invention for the preparation of a medicament for treating a disease or disorder in a subject. In one embodiment, the invention refers to the use of a pharmaceutical composition of the invention for treating a disease or disorder in a subject.

The term “subject” or “individual” are used interchangeably and may be, for example, a human or a non-human mammal. For example, the subject is a bat; a ferret; a rabbit; a feline (cat); a canine (dog); a primate (monkey), an equine (horse); a human, including man, woman and child. In one embodiment a “subject” refers to a human.

In the context of the invention, the term “treating” or “treatment”, refers to a therapeutic use (i.e. on a subject having a given disease) and means reversing, alleviating, inhibiting the progress of one or more symptoms of such disorder or condition. Therefore treatment does not only refer to a treatment that leads to a complete cure of the disease, but also to treatments that slow down the progression of the disease and/or prolong the survival of the subject.

By “preventing” is meant a prophylactic use (i.e. on a subject susceptible of developing a given disease).

A “disease” or “disorder” is any condition that would benefit from treatment with the antibody. This includes chronic and acute disorders or diseases including those pathological conditions which predisposes the subject to the disorder in question.

The term “in need of treatment” refers to a subject having already the disorder as well as those in which the disorder is to be prevented.

In one embodiment the disorder refers to a disorder in which TNFAlpha activity is detrimental.

As used herein, the term “a disorder in which TNFAlpha activity is detrimental” includes diseases and disorders in which the presence of TNFAlpha in a subject suffering from the disorder has been shown to be or is suspected of being either responsible for the pathophysiology of the disorder or a factor that contributes to a worsening of the disorder. Accordingly, a disorder in which TNFAlpha activity is detrimental is a disorder in which inhibition of TNFAlpha activity is expected to alleviate the symptoms and/or progression of the disorder. Such disorders may be evidenced, for example, by an increase in the concentration of TNFAlpha in a biological fluid of a subject suffering from the disorder (e.g., an increase in the concentration of TNFAlpha in serum, plasma, synovial fluid, etc. of the subject), which can be detected, for example, using an anti-TNFAlpha antibody as described above. There are numerous examples of disorders in which TNFAlpha activity is detrimental. Examples of disorders in which TNFAlpha activity is detrimental are described in U.S. Application No. 60/397,275, incorporated by reference herein. Examples in which TNFAlpha activity is detrimental are also described in U.S. Pat. Nos. 6,015,557, 6,177,077, 6,379,666, 6,419,934, 6,419,944, 6,423,321, and 6,428,787; U.S. Patent Application Nos. US2001/0016195, US2001/0004456, and US2001/026801; WO 00/50079 and WO 01/49321, each incorporated by reference herein.

In one embodiment the disease or disorders are plaque psoriasis, crohn's disease, ulcerative colitis, psoriatic arthritis, ankylosing spondylitis, rheumatoid arthritis, polyarticular and juvenile idiopathic arthritis.

In another embodiment, the disorder refers to a disorder associated with atypical or abnormal CXCR5 biology and function.

As used herein, the term “a disorder associated with atypical or abnormal CXCR5 biology and function” refers to a disorder which is characterized by or caused by overexpression or increased levels of CXCL13 or other CXCR5 ligand, increased levels of B cells, increased levels of B cell activity, increased levels of CXCR5 or improper metabolism and activity of CXCR5. Such disorders include autoimmune disorders such as lupus, Sjoren's syndrome, myasthenia gravis and multiple sclerosis; colitis, rheumatoid arthritis or psoriatic arthritis.

An “effective amount” refers to an amount effective, at dosages and for periods of time necessary, to achieve the desired therapeutic or prophylactic result.

A “therapeutically effective amount” of the pharmaceutical composition of the invention may vary according to factors such as the disease state, age, sex, and weight of the individual, and the ability of the antibody in context of the invention, to elicit a desired therapeutic result. A therapeutically effective amount encompasses an amount in which any toxic or detrimental effects of the antibody are outweighed by the therapeutically beneficial effects. A therapeutically effective amount also encompasses an amount sufficient to confer benefit, e.g., clinical benefit.

A physician or veterinarian having ordinary skill in the art can readily determine and prescribe the effective amount of the pharmaceutical composition of the present invention required. For example, the physician or veterinarian could start doses of the composition of the invention at levels lower than that required in order to achieve the desired therapeutic effect and gradually increase the dosage until the desired effect is achieved.

In adults, the dose of the composition may be for example 40 mg given every two weeks. For adults with for example Crohn's disease and for example psoriasis, an initial (induction) dose may be 80 mg followed by for example one week later by for example 40 mg every two weeks. For ulcerative colitis for example the first two doses are usually 160 mg and 80 mg given for example two weeks apart followed by for instance 40 mg every two weeks.

In one embodiment, the effective amount of the pharmaceutical composition is the amount necessary or sufficient to inhibit TNFAlpha activity, e.g. prevent the various morphological and somatic symptoms of a detrimental TNFAlpha activity-associated state.

The pharmaceutical composition is administered to a subject in accordance with known methods, such as intravenous administration, e.g., as a bolus or by continuous infusion over a period of time, by intramuscular, intraperitoneal, intracerobrospinal, subcutaneous, intra-articular, intrasynovial, or intrathecal administration, for instance by intramuscular or subcutaneous administration.

In one embodiment the administration is a subcutaneous administration. Therefore, in one embodiment of the present invention, said pharmaceutical composition is adapted for subcutaneous administration. In subcutaneous administration, or injection, of a drug the drug delivers a bolus into the subcutis the layer of skin directly below the dermis and epidermis, collectively referred to as the cutis. Subcutaneous injections are highly effective, and well established, in administering medications such as insulin, as they can be performed by non-medically skilled persons provided they have received respective training because of reduced risk of infection and ease of administration. Subcutaneous administration is thus suitable for ambulant administration, administration in areas of poor infrastructure, e.g., where non-medically skilled persons are responsible for the drug administration, or home use.

Subcutaneous administration is for example important in therapeutic regimens which require repeated treatment, as is the case in many chronic diseases, like autoimmune diseases (e.g., rheumatoid arthritis or ankylosing spondylitis) or in many cancer types which, due to targeted therapy, turn chronic or near-chronic.

However, it should be noted that for the above mentioned reasons pharmaceutical compositions which are adapted for subcutaneous administration have a higher risk to be exposed to suboptimal storage conditions by ordinary persons, e.g., the cool chain is interrupted, or the compositions are exposed to light or sudden temperature changes. Furthermore, subcutaneous administration compositions require a relatively high concentration of the therapeutic agent, because the volume administered with one injection is rather limited (0.1 to a maximum of 2.0 mL). In addition, in order to reduce needle pain during injection, the needle needs to be thin, requiring a low viscosity of the injected solution. And finally, subcutaneous injection may result in pain at the injection site, even after the needle has been removed. This is probably influenced by components of the protein solution, such as the sort of buffer molecules and the osmolarity, and may have a significant influence on subject compliance of the respective therapy. The composition according to the present invention, with its improved stability and improved subject compliance is thus suitable for subcutaneous administration.

For the prevention or treatment of disease, the appropriate dosage of the antibody will depend on the type of disease to be treated, as defined above, the severity and course of the disease, whether the monoclonal antibody is administered for preventive or therapeutic purposes, previous therapy, the subject's clinical history and response to the monoclonal antibody, and the discretion of the attending physician. The antibody is suitably administered to the subject at one time or over a series of treatments. Depending on the type and severity of the disease, about 1 μg/kg to 50 mg/kg (e.g. 0.1-20 mg/kg) of antibody is an initial candidate dosage for administration to the subject, whether, for example, by one or more separate administrations, or by continuous infusion. The dosage of the antibody will generally be from about 0.05 mg/kg to about 10 mg/kg.

If another therapeutic agent is administered, it is usually administered at dosages known therefore, or optionally lowered due to combined action of the drugs or negative side effects attributable to administration of the therapeutic agent. Preparation and dosing schedules for such therapeutic agents may be used according to manufacturers' instructions or as determined empirically by the skilled practitioner.

### Articles of Manufacture

The invention herein also concerns a device comprising a pharmaceutical composition of the invention. Such devices may hold a liquid volume of between 0.1 ml and 2 ml (single use), or 0.5 and 1.5 ml. In one embodiment the volume is about 0.8 or about 1.0 ml.

In one embodiment the device is for subcutaneous delivery. For subcutaneous delivery, the composition may be administered via syringe (e.g. pre-filled syringe); auto injector; injection device (e.g. the INJECT-EASE™ and GENJECT™ device); injector pen (such as the GENPEN™); or other device suitable for administering a suspension composition subcutaneously. In one embodiment the device herein is a pre-filled syringe.

In a related aspect, the invention provides a method of making an article of manufacture comprising filling a container with the pharmaceutical composition of the invention.

Embodiments of the container in the article of manufacture include: syringes (such as pre-filled syringe), auto injectors, bottles, vials (e.g. dual chamber vials), and test tubes, etc. The container holds the suspension composition and the label on, or associated with, the container may indicate directions for use. The article of manufacture may further include other materials desirable from a commercial and user standpoint, including other buffers, diluents, filters, needles, syringes, and package inserts with instructions for use as noted in the previous section.

### Kit

According to another aspect of the present invention, a kit is provided; said kit comprises at least one container comprising at least one pharmaceutical composition as described above, and an injection device. In one embodiment the kit or the injection device is adapted for intramuscular or subcutaneous administration, for instance for subcutaneous administration. In one embodiment the kit further comprises instructions for administration of the composition, for example for subcutaneous administration.

According to yet another embodiment of the present invention, the use of a device comprising the pharmaceutical composition or of a kit according to the invention is provided. In a further embodiment the invention refers to the use of the device comprising the pharmaceutical composition of the invention or of a kit in context of the invention for treatment of at least one disease as described above.

### Method for Reducing Aggregation

In view to the above, the present invention also relates to a method for reducing aggregation and/or fragmentation of an antibody by using a composition according to the present invention. A skilled person will understand that formulating a therapeutically active antibody in a composition according to the present invention which is susceptible to aggregation or less stable will lead to a reduced amount of aggregation and stabilization of the antibody compared to a reference composition.

Thus, in one aspect, the present invention relates to a method for reducing aggregation of an antibody, comprising formulating an antibody in a composition comprising:

b) at least one buffer agent selected from a group consisting of acetate or histidine,
c) at least one amino acid wherein the amino acid is selected from the group consisting of glycine, asparagine and glutamine, for example glycine and asparagine, and/or at least one excipient selected from the group consisting of threhalose and mannitol, and
d) a surfactant,
wherein the pH is 5.0 to 6.5.

In a further aspect the invention refers to a method of stabilizing an antibody comprising formulating an antibody in a composition of the invention.

An antibody which is “susceptible to aggregation” has been found to aggregate with other antibody molecule(s), especially upon freezing, stirring and/or at increased temperatures such as 40° or 55° C. An antibody that is susceptible to aggregation might be for example an antibody that has less than 94%, less than 93%, less than 92%, less than 90% of monomer after storage at about 55° C. for one week as measured by SEC. An antibody that is susceptible to aggregation might be for example an antibody that has less than 94%, less than 93%, less than 92%, less than 90% of monomer after storage at about 40° C. for 3 months as measured by DLS.

An antibody which is “susceptible to fragmentation” is one which has been found to be cleaved into two or more fragments, for example at a hinge region thereof.

By “reducing, aggregation, or fragmentation” is intended preventing or decreasing the amount of, aggregation, or fragmentation relative to the antibody formulated in a reference composition such as the market formulation of Humira®.

Any combination of the above embodiments makes part of the invention.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”). Furthermore the indefinite article “a” or “an” does not exclude a plurality. The mere fact that certain measures are recited in mutually different dependent claims does not indicate that a combination of these measures cannot be used to advantage.

The invention will now be described in more detail with reference to the following examples. All literature and patent documents cited herein are hereby incorporated by reference. While the invention has been illustrated and described in detail in the the foregoing description, the examples are to be considered illustrative or exemplary and not restrictive.

### BRIEF DESCRIPTION OF THE SEQUENCES

SEQ ID NO: 1 shows the heavy chain sequence of the anti-TNFAlpha antibody.

SEQ ID NO: 2 shows the light chain sequence of the anti-TNFAlpha antibody.

### EXAMPLES

### 1. Methods

### 1.1 Sample Preparation

Different buffers were initially screened using the anti-TNFAlpha antibody. This was done by dialyzing the anti-TNFAlpha antibody against the buffers listed in Table 1 using viva spin (15R; Membran: 30,000 MWCO HY). For comparison the buffer screen was also performed for the commercially available Adalimumab antibody.

TABLE 1
Buffer and pH values screened during anti-TNFAlpha
antibody phase III composition development study

| Buffer | pH values |
|---|---|
| Tris-citrate | 5.0, 5.5, 6.0, 6.5, 7.0 |
| Tris | 7.0 |
| Phosphate | 6.5, 7.0 |
| Histidine | 5.5, 6.0, 6.5 |
| Citrate | 5.0, 5.5, 6.0 |
| Acetate | 5.0, 5.5 |
| Succinate | 5.0, 5.5, 6.0 |
| Phosphate-citrate | 5.2, 5.5, 6.0 |

For the excipient screen as described in 2.1.6, excipients such as sucrose, trehalose, mannitol, sorbitol, glycerol, L-argenine HCl, L-glycine, L-aspargine monohydrate, L-glutamine, L-glutamic acid, sodium chloride, polysorbate 20 (PS 20) and polysorbate 80 (PS 80) were either added to the anti-TNFAlpha antibody in powder form (e.g. mannitol and sodium chloride) or added in liquid form (e.g. Tween 80 as stock solution). All samples, solutions and buffers were sterile filtered (0.22 μm) using a Sartopore-2 membrane. The samples were filtered into sterilized bottles or vials, closed under aseptic conditions inside a clean-bench to prevent microbiological contamination.

### 1.2 Analytical Methods

Differential scanning microcalorimetry (μDSC) was used in most screening studies (buffer screening, ionic strength, excipients screening) in order to make a pre-selection before entering into stress studies. Compositions that entered stress studies (that are further described in section 1.3) were then analyzed using analytical methods such as for example size exclusion chromatography (SEC), weak cationic exchange chromatography (WCX), Light blockage and/or dynamic light scattering (DLS) or further analytical methods described in the following.

### 1.2.1 Differential Scanning Microcalorimetry (μDSC)

Differential scanning microcalorimetry (μDSC) was used in all screening studies in order to make a pre-selection before entering into stress studies. All measurements were performed using a VP Capillary μDSC from GE Healthcare. Basically, each sample was heated from 30-100° C. using 90° C./hr heating rate. Mainly the unfolding temperature (Tm) was used as a parameter to select the most promising compositions. In particular, the Tm of the Fc1 domain was used as parameter for the ranking, because it was the first unfolding event in the thermogram.

### 1.2.2 Size Exclusion Chromatography (SEC)

Size exclusion chromatography (SEC) was used to determine the relative amount of monomer as well as high molecular weight variants (HMW) and low molecular weight variants (LMW). Size exclusion chromatography is used for protein separation according to the size of the antibody, soluble aggregates (HMW) and antibody fragments (LMW). Aggregates elute prior to the intact antibody and fragments dilute after. The percentage area of the main peak in relation to the total area in percentage of all peaks is used for the evaluation.

### 1.2.3 Weak Cationic Exchange Chromatography (WCX)

The level of charged isoforms was measured using weak cationic exchange chromatography. The chromatographic separation is carried out on a weak cationic exchange column coupled to UV detection. Weak cationic exchange chromatography (WCX) separates different isoforms of the antibody based on charge heterogeneity. More acidic isoforms show less ionic interactions and therefore elute earlier in the WCX chromatogram than basic isoforms The aim of this method is to determine the relative amount of charged isoform. The sum of acidic, neutral, and basic isoforms is set 100% and the main peak area, the area of the acidic isoforms and that of the basic isoforms is calculated relatively.

### 1.2.4 Dynamic Light Scattering (DLS):

The presence of aggregates and particles in the nanometer size range were measured using Zetasizer Nano-ZS from Malvern. The particle size distribution was measured by intensity and by volume. Additionally the hydrodynamic diameter and poly-disperse index was measured. For this measurement 250-300 μl were needed.

### 1.2.5 Light Blockage/Light Obscuration (LO)

Light obscuration measurements were performed to evaluate the size and concentration of sub visible particles in compositions containing anti-TNFAlpha antibodies. The measurements were performed using HIAC® particle counter (HACH LANGE, Dusseldorf, Germany). For this measurements 800-1000 μl were needed

### 1.2.6 Microflow Imaging

This method was used in some cases to look more deeply to the form of the particles formed. In other cases, this method was used as an alternative method to the light blockage.

### 1.2.7 Differential Scanning Fluoremetry (DSF)

The measurements were performed using CFX96 BioRad. The temperature scanning ranged from 20° C. to 90° C. using a heating rate 1° C./minute and SyproOrange fluorescence reporting dye: Invitrogen, diluted in water, 5× final concentration. 21 different compositions were tested against placebo at a concentration of 5 mg/ml. 9 μl samples were added to 1 μl SyproOrange fluorescence dye which results in 4.5 mg/ml final anti-TNFAlpha antibody concentration. Each composition was measured twice.

1.8 2^{nd} Viral Osmotic Coefficient (B₂₂)

The osmotic second virial coefficient, A₂ or B₂₂, is a measure of the protein-protein interaction as well as the protein-solvent interaction. Virial coefficients indicate the overall attraction or repulsion between molecules, providing a general measure of the intermolecular potential as mediated by the solvent. In biotechnology applications, virial coefficients can help determine optimal conditions for stability, purification, and crystallization of compositions by evaluating changes in pH, ionic strength, and concentrations of various excipients in the buffer. The B₂₂ value is calculated from the slope of the Debye plot (K_{c}/R^{θ} versus c) generated from a range of concentrations of each composition using static light scattering methodology and is reported in mol mL g⁻². A positive value of B₂₂ indicates more repulsion and thus less aggregate formation and a negative B₂₂ values indicates more attractive forces and thus more aggregation tendency. Accordingly a higher positive B₂₂ is favorable.

### 1.3 Stress Studies

### 1.3.1 Accelerated Stability Studies

Different accelerated stability studies were applied in this study wherein the compositions were exposed to different kind of stress.

Mechanical stress: Mechanical stress according to the present study was stirring for example at 200 rpm for max 6 hrs. This condition was adjusted accordingly in each experiment. In this study the samples were stressed using stirring in vials, herein a Variomag Multipoint HP stirrer was used.

Short isothermal stress: In the short isothermal stability studies samples were stored at 40° C. for 7-14 days according to the test applied.

Long isothermal stress: In the long isothermal stability studies samples were stored at 40° C. for 1, 3 or 6 months according to the test applied.

Accelerated thermal stress: In the accelerated stability studies samples were stored at 50 and 55° C. for up to 1 month. These conditions were for example applied to the final selection of different compositions.

Freezing and thawing: In this study samples were frozen at −80° C. for 24 hrs and thawn at room temperature for 90 min. This was repeated for 5 cycles. The 5^{th} cycle was kept at −80° C. for 72 hrs.

### 1.3.2 Exploratory Stability Studies

For a final selection of a final composition longer isothermal stability studies were performed with compositions of the invention. Therefore, the compositions comprising anti-TNFAlpha antibodies were stored up to 6 months at different temperatures (−80, −20, 5, 25, and 40° C.). Then the samples were checked for stability.

### 1.4 Composition Selection Method/Ranking

To avoid misleading interpretation of the different analytical methods mentioned above under section 1.2, a ranking method was established based on the significance of each method. A first ranking was performed within a group of compositions according to the results obtained from each analytical method in each stability study. The final ranking is then obtained by calculating the average of the different rankings obtained from all analytical methods. According to the stress applied, the importance of each analytical method was evaluated and considered in the ranking procedure.

### 1.4.1 Physical Stability Ranking

The total physical stability ranking was calculated based on the average ranking from all the methods mentioned below. The importance of the methods changed according to the stress applied. For example for storage stress the SEC ranking was considered more important than the particle analysis and for mechanical stress as well as stress derived from Freeze and Thaw the particle analysis was considered more important than SEC.

Size exclusion chromatography (SEC): The different compositions have all stabilizing effects, therefore a difference of only 0.5% of the monomer content was considered within this ranking as significantly different.

Dynamic light scattering (DLS): 4 ranking parameters were used: The first ranking parameter is the monomer content analyzed by volume (1^{st}), the 2nd ranking parameter is the monomer content analyzed by intensity (2^{nd}), the Z-average is the 3^{rd} ranking parameter and the 4th ranking parameter is the polydispersity index (PDI). The ranking is done in the above mentioned order in a way that when a composition failed in the 1st it will not be further considered in the rest and similarly for the 2^{nd}, 3^{rd}, 4^{th} ranking parameters.

Light obscuration (LO): The compositions were ranked according to the size distributions observed within the composition. The composition having less than 6000 particles >10 μm, less than 600 particles >25 μm and less than 10000 particles >1 μm were ranked 1. All compositions having less than 6000 particles >10 μm, less than 600 particles >25 μm and less than 100000 particles >1 μm were ranked 2. All composition having less than 6000 particles >10 μm, less than 600 particles >25 μm and more than 100000 particles >1 μm were ranked 3. All composition that breaks the limits for particles >10 μm and >25 μm regardless the particle count >1 μm were ranked 4.

Color and clarity: The ranking was simply based on the Formazin Nephelometry units (FNU) units and a significant change refers to a FNU change that leads to a change in the standard Number (e.g. from <I to <II).

### 1.4.2 Chemical Stability Ranking

Weak cationic exchange chromatography (WCX): The change in the percentage of acidic, neutral and basic isoforms was the ranking parameter. A change of 2-3% is considered as significant.

### 1.4.3 Overall Stability Ranking

The overall stability ranking results from the average of both physical and chemical stability ranking, wherein both, physical and chemical stability were considered of equal importance.

### 2 Results and Discussion

### 2.1 Buffer Screening (Example 1)

### 2.1.1 Initial Buffer Screen

The stability of Adalimumab and its biosimilar (herein called anti-TNFAlpha antibody BS) was tested in different buffers (10 mM) and pH values. For analysis differential scanning microcalorimetry (μDSC) was used as analytical method as described above in section 1.2.1. Anti-TNFAlpha antibody BS unfolding curves showed the presence of 4 domains that unfold independently (Fab, Fc1, Fc2, Fc3), the peak having the largest enthalpy is the Fab Fragment and the other 3 are Fc Fragments (data not shown). For ranking purposes the Fc1 fragment peak was used as this is the first unfolding event that occurs. Anti-TNFAlpha antibody BS and Adalimumab gave similar results (similar T_{M} values). The optimum pH range was determined as pH 5.5 to 6.5 for both molecules in most buffers. Table 2 shows the unfolding temperatures obtained for the anti-TNFAlpha antibody BS.

TABLE 2
T_{M} values for anti-TNFAlpha antibody BS in different buffer systems.

| | | Unfolding temperatures | | | |
|---|---|---|---|---|---|
| | | (Tm) of different domains | | | |
| Buffer¹ | pH value | Fab | Fc1 | Fc2 | Fc3 |
| Tris-Citrate | 5 | 71.18 | 64.88 | 73.13 | 81.87 |
| Tris-Citrate | 5.5 | 71.73 | 71.09 | 76.24 | 82.58 |
| Tris-Citrate | 6 | 71.91 | 70.25 | 74.08 | 83.34 |
| Tris-Citrate | 7 | 72 | 71.77 | 73.48 | 83.48 |
| Tris | 7.1 | 72.78 | 72.08 | 77.46 | 83.82 |
| Phosphate | 6.5 | 72.76 | 72.68 | 76.82 | 83.59 |
| Phosphate | 7 | 72.59 | 68.28 | 75.58 | 83.26 |
| Histidine | 5.5 | 71.93 | | | |
| Histidine | 6 | 73.03 | 71.28 | 76.28 | 83.3 |
| Histidine | 6.5 | 73.36 | 71.57 | 74.08 | 83.93 |
| Citrate | 5 | 70.7 | 65.55 | 74.55 | 81.53 |
| Citrate | 5.5 | 71.3 | 70.06 | 75.83 | 83.12 |
| Acetate | 5 | 72.74 | 69.27 | 75.86 | 83.25 |
| Acetate | 5.5 | 72.99 | 72.4 | 79.72 | 84.08 |
| Succinate | 5 | 71.94 | 67.38 | 74.98 | 82.72 |
| Succinate | 5.5 | 72.3 | 71.1 | 76.48 | 83.25 |
| Succinate | 6 | 72.68 | 71.41 | 73.59 | 83.47 |
| Citrate-Phosphate | 5.2 | 71.59 | 68.7 | 75.29 | 82.24 |
| Original | 5.5 | 71.77 | 70.24 | 75.98 | 82.83 |
| Original | 6 | 72.08 | 70.45 | 74.26 | 83.41 |
| Citrate-Phosphate + Exc². | 5.2 | 71.52 | 68.8 | 75.12 | 82.15 |

¹in 10 mM buffer,
²Exc. = Excipients of Humira ® formulation

The 8 best buffer systems were selected and a mechanical stress study was performed to select the best buffer systems. Additionally the originator buffer and the originator composition (market formulation) were tested as references. Mechanical stability studies were chosen as an accelerated stability study to monitor if the buffer and pH value can improve the mechanical stability of anti-TNFAlpha antibody because mechanical instability was determined to be the main week point of said antibodies. To monitor mechanical stability the analytical techniques such as size exclusion chromatography (SEC), light obscuration (LO) and dynamic light scattering (DLS) were chosen (as described in section 1.2). The results of the mechanical stress study were evaluated based on the ranking system described in section 1.3 and are shown in table 3.

TABLE 3
Final ranking after mechanical stability studies of the different
buffer compositions for anti-TNFAlpha antibody BS

| | Rank | Rank | Rank | | |
|---|---|---|---|---|---|
| Composition¹ | SEC | DLS | HIAC | Average | Rank |
| Phosphate pH 6.5 | 6 | 9 | 2 | 5.666667 | 6 |
| Acetate pH 5.5 | 1 | 1 | 3 | 1.666667 | 1 |
| Histidine pH 6 | 4 | 2 | 3 | 3 | 2 |
| Tris Citrate pH 5.5 | 7 | 4 | 6 | 5.666667 | 6 |
| Tris Citrate pH 6 | 8 | 8 | 3 | 6.333333 | 8 |
| Tris Citrate pH 6.5 | 9 | 7 | 6 | 7.333333 | 9 |
| Tris Citrate pH 7 | 10 | 10 | 6 | 8.666667 | 10 |
| Succinate pH 6 | 5 | 3 | 6 | 4.666667 | 4 |
| Citrate-Phosphate pH 5.2 | 2 | 6 | 6 | 4.666667 | 4 |
| Citrate-Phosphate + | 3 | 5 | 1 | 3 | 2 |
| Exc.² pH 5.2 | | | | | |

¹in 10 mM buffer
²Exc. = Excipients of Humira ® formulation

According to this ranking acetate buffer at pH 5.5 and histidine buffer at pH 6 showed the best mechanical stability for the tested antibody and were selected for further stability studies. Additionally, tris-citrate buffer at pH 6 was selected in order to have a combined buffer in the stability study.

### 2.1.2 DS Stability Study

The selected three buffers (acetate buffer at pH 5.5, histidine buffer at pH 6 and tris-citrate buffer at pH 6) were further tested in a short exploratory stability study at −80, −20, 5, 25, and 40° C. (see section 1.3.2). A Pre-selection of the final buffer was made after 1 month (data not shown) and was confirmed after 3 months (data not shown). The study was further analyzed after 6 months. For this study for analysis the analytical methods SEC, WCX, DLS, UV, appearance, LO, SDS-PAGE and ELISA were used. Based on the results 10 mM acetate buffer pH 5.5 was selected to be the final buffer composition for anti-TNFAlpha antibody BS. As a back-up histidine buffer at pH 6 was selected as a second choice. Tris-citrate buffer at pH 6 didn't show benefit over the other buffer system selected and therefore was excluded due to a low mechanical stability of the anti-TNFAlpha antibody BS observed in the mechanical stability studies shown in table 3.

### 2.1.4 Surfactant Screening (Example 2)

Both PS 20 and PS 80 were initially tested in three different concentrations (0.001% w/v, 0.01% w/v and 0.1% w/v) in acetate buffer pH 5.5. As the main expected effect of the surfactants is to protect the anti-TNFAlpha antibody BS against mechanical stress, only stirring experiments were performed as described under section BS. Samples were analyzed by: SEC, WCX, Light obscuration, and DLS. Based on the results shown in table 4 it can be concluded that 0.01% w/v PS 20, 0.1% w/v PS 80 and 0.01% w/v PS 80 are the optimal concentration.

TABLE 4
Polysorbate mechanical stability final ranking

| Composition¹ | Rank SEC | Rank DLS | Rank HIAC | Average | Rank |
|---|---|---|---|---|---|
| No PS | 1 | 1 | 7 | 3 | 6 |
| PS 20 0.001% | 1 | 1 | 6 | 2.67 | 4 |
| PS 20 0.01% | 1 | 1 | 1 | 1 | 1 |
| PS 20 0.1% | 1 | 7 | 1 | 3 | 7 |
| PS 80 0.001% | 1 | 1 | 5 | 2.33 | 5 |
| PS 80 0.01% | 1 | 1 | 1 | 1 | 1 |
| PS 80 0.1% | 1 | 1 | 1 | 1 | 1 |

¹in acetate buffer pH 5.5

However, there is a trend to have fewer particles at the higher concentration of PS 80 at 0.1%. Accordingly, for further testing of excipient combinations PS 20 at 0.01% w/v (0.1 mg/ml) or PS 80 at 0.1% w/v (1 mg/ml) were considered.

### 2.1.5 Ionic Strength of the Composition (Example 3)

The ionic strength of the composition was tested in term of different buffer concentrations and different NaCl concentrations. μDSC screening was made for 10 and 100 mM buffer concentrations and for 2 and 20 mg/ml NaCl concentrations. The resulting unfolding temperatures are listed in table 5.

TABLE 5
Unfolding Temperatures of anti-TNFAlpha antibody BS at different ionic
strengths

| | | | Unfolding temperatures (Tm) | | | |
|---|---|---|---|---|---|---|
| | Conc. | NaCl | of different domains | | | |
| Buffer (pH value) | (mM) | (mg/mL) | Fab | Fc1 | Fc2 | Fc3 |
| Acetate (pH 5.5) | 10 | 0 | 73.44 | 73.53 | 82.01 | 84.61 |
| Tris-citrate (pH 6) | 10 | 0 | 71.9 | 69.47 | 74.28 | 83.34 |
| Histidine (pH 6) | 10 | 0 | 73.78 | 71.67 | 82.91 | 85.11 |
| Acetate (pH 5.5) | 100 | 0 | 72.34 | 70.11 | 74.82 | 82.53 |
| Tris-citrate (pH 6) | 100 | 0 | 72.44 | 71.02 | 72.25 | 82.75 |
| Histidine (pH 6) | 100 | 0 | 72.34 | 68.84 | 74.87 | 82.65 |
| Acetate (pH 5.5) | 10 | 2 | 72.61 | 71.94 | 76.11 | 83.17 |
| Tris-citrate (pH 6) | 10 | 2 | 72.15 | 71.42 | 76 | 83.34 |
| Histidine (pH 6) | 10 | 2 | 72.51 | 70.35 | 74.90 | 82.76 |
| Acetate (pH 5.5) | 10 | 20 | 71.14 | 67.12 | 73.79 | 82.26 |
| Tris-citrate (pH 6) | 10 | 20 | 71.54 | 68.29 | 74.53 | 82.48 |
| Histidine (pH 6) | 10 | 20 | 71.46 | 68.28 | 74.2 | 82.13 |

The Tₘ screening study shows that the higher the buffer concentration is the lower is the unfolding temperature except in the case of tris-citrate buffer. Additionally, adding NaCl does not favor the stability of the anti-TNFAlpha antibody BS as concluded from the obtained Tₘ values. Furthermore increasing the concentration of NaCl has a strong negative effect on Tₘ. As described above, after 1 month data of the DS stability study described in 2.1.2, acetate buffer was selected as the preferred buffer for the compositions according to the invention and therefore the accelerated stability study was performed using acetate buffer. Short isothermal stability studies (40° C. for 7 days) and mechanical stability (200 rpm stirring for 2 hours) studies were then performed. Samples were analyzed before and after stress using the following analytical techniques: SEC, WCX, Light blockage, and DLS.

For the accelerated stability study lower buffer concentrations of 5 mM were compared with 10 mM buffer concentration in presence and absence of 2 mg/ml NaCl. Table 6 shows the final ranking of the compositions after thermal stress and mechanical stress. The final ranking as shown in table 6 shows better stability for a buffer concentration of 10 mM and no significant difference for NaCl concentration of 0 to 2 mg/ml. Accordingly the buffer concentration is fixed to 10 mM and the presence and absence of NaCl (2 mg/ml) was considered later on for excipient combination study.

TABLE 6
Ionic strength stability general ranking (physical and chemical)

| | Conditions | | | | | |
|---|---|---|---|---|---|---|
| | Time | | Physi- | Chemi- | | Average |
| Composition | point | stress | cal | cal | Rank | Ranking |
| Acetate buffer | T0 | / | 1 | 1 | 1 | 1.22 |
| 10 mM | 1 week | 40° C. | 1 | 1 | 1 | |
| | 1 hour | 200 rpm | 2.33 | 1 | 1.67 | |
| Acetate buffer | T0 | / | 1.33 | 1 | 1.17 | 1.22 |
| 10 mM and | 1 week | 40° C. | 2 | 1 | 1.5 | |
| 2 mg/mL NaCl | 1 hour | 200 rpm | 1 | 1 | 1 | |
| Acetate buffer | T0 | / | 3.33 | 1 | 2.17 | 1.89 |
| 5 mM | 1 week | 40° C. | 2.67 | 1 | 1.83 | |
| | 1 hour | 200 rpm | 2.33 | 1 | 1.67 | |
| Acetate buffer | T0 | / | 3 | 1 | 2 | 1.78 |
| 5 mM and | 1 week | 40° C. | 3.67 | 1 | 2.33 | |
| 2 mg/mL NaCl | 1 hour | 200 rpm | 1 | 1 | 1 | |

### 2.1.6 Excipients Screening (Example 4):

### Sugars and Polyols:

Different sugars and polyols were tested in acetate buffer (10 mM, pH 5.5) for their effect on anti-TNFAlpha antibody BS stability using μDSC. The results of the study are shown in table 7. Trehalose and mannitol showed the highest unfolding temperatures. The ranking here (and also in all μDSC screening studies) was based on the Tm of the Fc1 domain as it was the first unfolding event in the thermogram. Sucrose, sorbitol and glycerol showed the lowest Tₘ values.

TABLE 7
Unfolding temperatures of compositions comprising different sugars and
polyols

| | | | Unfolding temperatures (Tm) of | | | |
|---|---|---|---|---|---|---|
| Composition¹ | Conc. | Conc. | different domains | | | |
| (Suger or Polyol) | (mM) | (mg/mL) | Fab | Fc1 | Fc2 | Fc3 |
| Sucrose | 146 | 50 | 73.65 | 72.36 | 74.85 | 83.95 |
| Trehalose | 146 | 55.24 | 73.76 | 73.77 | 79.52 | 84.2 |
| Mannitol | 146 | 26.6 | 73.52 | 73.4 | 79 | 83.94 |
| Sorbitol | 146 | 26.6 | 73.54 | 72.15 | 74.66 | 83.75 |
| Glycerol | 146 | 13.35 | 73.17 | 72.94 | 77.15 | 83.52 |

¹in 10 mM acetate buffer pH 5.5

Accordingly, mannitol and trehalose were tested in acetate buffer (10 mM, pH 5.5) in 2 different concentrations in accelerated stability studies (short isothermal stability study (40° C. for 7 days) and mechanical stability studies (200 rpm stirring for 2 hours). Additionally, sorbitol was tested in the acceleration study as a negative control in order to verify the selection based on Tₘ value. Samples submitted to the accelerated stability study were analyzed before and after stress using the following analytical techniques: SEC, WCX, Light blockage, and DLS. The resulting final ranking showed better stability for mannitol as excipient as shown in table 8. Accordingly, different concentrations of mannitol were selected for further studies.

TABLE 8
Sugar and polyols stability general ranking (physical and chemical)

| | Conditions | | | | | |
|---|---|---|---|---|---|---|
| | Time | | | Chemi- | | Average |
| Composition¹ | point | stress | Physical | cal | Rank | Ranking |
| Mannitol 10 mg/ml | T0 | / | 1 | 1 | 1 | 1.375 |
| | 1 W | 40° C. | 1.75 | 1 | 1.375 | |
| | 1 hr | 200 rpm | 2.5 | 1 | 1.75 | |
| Mannitol 40 mg/ml | T0 | / | 2 | 1 | 1.5 | 1.708 |
| | 1 W | 40° C. | 3.25 | 1 | 2.125 | |
| | 1 hr | 200 rpm | 2 | 1 | 1.5 | |
| Sorbitol 40 mg/ml | T0 | / | 1.5 | 1 | 1.25 | 1.875 |
| | 1 W | 40° C. | 3.25 | 2 | 2.625 | |
| | 1 hr | 200 rpm | 2.5 | 1 | 1.75 | |
| Trehalose 50 mg/ml | T0 | / | 1 | 1 | 1 | 1.958 |
| | 1 W | 40° C. | 1 | 3 | 2 | |
| | 1 hr | 200 rpm | 1.75 | 4 | 2.875 | |
| Trehalose 20 mg/ml | T0 | / | 1.5 | 1 | 1.25 | 2.208 |
| | 1 W | 40° C. | 1.75 | 3 | 2.375 | |
| | 1 hr | 200 rpm | 2 | 4 | 3 | |

¹in 10 mM acetate buffer pH 5.5

### Amino Acids:

Furthermore, different amino acids were tested in 10 mM acetate buffer (pH 5.5) using μDSC. The resulted Tₘs are listed in table 9.

TABLE 9
Unfolding Temperatures of anti-TNFAlpha antibody BS for different
amino acids as excipients

| | | | Unfolding temperatures | | | |
|---|---|---|---|---|---|---|
| Composition¹ | Conc. | Conc. | (Tm) of different domains | | | |
| (Amino Acid) | (mM) | (mg/mL) | Fab | Fc1 | Fc2 | Fc3 |
| Arginine | 200 | 42.13 | 70.48 | 68.09 | 74.35 | 81.89 |
| Glycine | 200 | 15 | 73.41 | 72.8 | 78.71 | 84.17 |
| L-lysine HCl | 200 | 36.53 | 71.14 | 69.46 | 75.27 | 82.76 |
| L-asparagine | 200 | 10 | 72.85 | 72.04 | 77.48 | 83.84 |
| Glutamine | 200 | 29.23 | 73.19 | 72.55 | 78.7 | 84.03 |
| Arginine/Glutamic | 100/100 | 17.42/14.71 | 72.18 | 70.66 | 76.52 | 83.55 |
| acid | | | | | | |

¹in 10 mM acetate buffer pH 5.5

The Tₘ screening study showed that glycine, L—asparagine and glutamine showed the highest Tₘ. On the other hand, arginine, L—lysine and arginine/glutamic acid showed the lowest Tₘ values. For the accelerated stability study glycine, L—asparagine and glutamine were tested in 2 different concentrations. The final ranking shown in table 10 demonstrates better stability when using glycine and asparagine.

TABLE 10
Amino acids stability general ranking (physical and chemical)

| | Conditions | | | | | |
|---|---|---|---|---|---|---|
| | Time | | Physi- | Chemi- | | Average |
| Composition¹ | point | stress | cal | cal | Rank | Ranking |
| Glycine | T0 | / | 1.25 | 1 | 1.125 | 1.333 |
| 7.5 mg/ml | 1 W | 40° C. | 1 | 2 | 1.5 | |
| | 1 hr | 200 rpm | 1.75 | 1 | 1.375 | |
| Glycine | T0 | / | 2.75 | 1 | 1.875 | 2.25 |
| 30 mg/ml | 1 W | 40° C. | 3.75 | 2 | 2.875 | |
| | 1 hr | 200 rpm | 3 | 1 | 2 | |
| Asparagine | T0 | / | 1 | 1 | 1 | 1.958 |
| 2 mg/ml | 1 W | 40° C. | 1 | 3 | 2 | |
| | 1 hr | 200 rpm | 1.75 | 4 | 2.875 | |
| Asparagine | T0 | / | 2.25 | 1 | 1.625 | 2.375 |
| 5 mg/ml | 1 W | 40° C. | 1 | 3 | 2 | |
| | 1 hr | 200 rpm | 3 | 4 | 3.5 | |
| Glutamine | T0 | / | 3.75 | 1 | 2.375 | 2.958 |
| 15 mg/ml | 1 W | 40° C. | 4 | 1 | 2.5 | |
| | 1 hr | 200 rpm | 4 | 4 | 4 | |
| Glutamine | T0 | / | 3.75 | 1 | 2.375 | 2.75 |
| 29.23 mg/ml | 1 W | 40° C. | 4.25 | 2 | 3.125 | |
| | 1 hr | 200 rpm | 4.5 | 1 | 2.75 | |

¹in 10 mM acetate buffer pH 5.5

### 2.2 Prototype Composition Selection (Example 5):

### 2.2.1 Design of Experiment (DOE)

After selection of the best conditions (Buffer, pH value and ionic strength) and the best stabilizers, a design of experiment (DOE) approach was used in order to select the final compositions. High throughput predictive methods such as differential scanning microcalorimetry (μDSC), differential scanning fluoremetry (DSF) and 2^{nd} viral osmotic coefficient (B₂₂) as described above in section 1.2 were used. The compositions included in the DOE and the results of each predictive method are presented in table 11.

TABLE 11
DOE compositions¹ and the results of three predictive high throughput
methods

| | | | | | μDSC | | |
|---|---|---|---|---|---|---|---|
| | NaCl | Mannitol | Glycine | | Fc1 | DSF | |
| Run | (mg/mL) | (mg/mL) | (mg/mL) | PS² | Tₘ | Tₘ | B₂₂ |
| 1 | 2 | 10 | 7.5 | 80 | 71.115 | 69.8 | |
| 2 | 2 | 10 | 7.5 | 20 | 71.58 | 64.9 | |
| 3 | 0 | 20 | 7.5 | 20 | 72.615 | 67 | |
| 4 | 2 | 20 | 15 | 80 | 71.48 | 70.4 | |
| 5 | 0 | 10 | 15 | 80 | 73.515 | 71.7 | |
| 6 | 0 | 0 | 15 | 20 | 73.685 | 66.8 | 6.59 |
| 7 | 4 | 20 | 0 | 20 | 70.475 | 64 | |
| 8 | 4 | 0 | 15 | 80 | 70.58 | 70 | |
| 9 | 4 | 0 | 7.5 | 20 | 70.54 | 64.3 | |
| 10 | 2 | 10 | 7.5 | 80 | 70.875 | 69.8 | 3.1 |
| 11 | 2 | 10 | 7.5 | 20 | 71.14 | 64.8 | |
| 12 | 4 | 10 | 0 | 80 | 70.075 | 69 | |
| 13 | 4 | 20 | 15 | 20 | 71.17 | 64.8 | 2.79 |
| 14 | 0 | 20 | 0 | 80 | 71.945 | 70.7 | 5.83 |
| 15 | 0 | 0 | 0 | 20 | 71.59 | 66.2 | |
| 16 | 2 | 0 | 0 | 80 | 70.37 | 69.4 | |
| 17 | 0 | 0 | 7.5 | 80 | 72.705 | 71 | |
| 18 | 4 | 20 | 7.5 | 80 | 70.765 | 69.8 | |
| 19 | 2 | 10 | 7.5 | 80 | 71.05 | 69.9 | |
| 20 | 2 | 10 | 7.5 | 20 | 71.12 | 64.9 | |
| 21 | 4 | 0 | 0 | 80 | 70.015 | 68.8 | 2.88 |

¹All composition are in 10 mM acetate buffer at pH 5.5
²PS 20 concentration is 0.1 mg/ml and PS 80 is 1 mg/ml

The results obtained from the DOE by μDSC showed: i) a significant negative effect of NaCl, ii) a significant positive effect of glycine, iii) a positive effect of mannitol iv) no difference between PS20 and PS80.

The results obtained from the DOE by DSF showed: i) a significant negative effect of NaCl, ii) a significant positive effect of mannitol, iii) a significant positive effect of PS 80 and iv) a significant negative effect of PS 20.

The results obtained by B₂₂ according to the DOE showed a significant negative effect of NaCl.

According to the statistical DOE, based on μDSC Tₘ and DSF Tₘ, 2 compositions were selected for the exploratory stability study together with additionally 2 compositions one with NaCl and another with only glycine as shown in table 12.

TABLE 12
Composition of the new developed 4 prototype compositions

| | | | | Glycine | |
|---|---|---|---|---|---|
| Buffer | | Mannitol | | (mg/ | NaCl |
| (mM) | pH | (mg/mL) | PS | mL) | (mg/mL) |
| Acetate (10 mM) | 5.5 | 20 | 80 (1 mg/ml) | 15 | 0 |
| Acetate (10 mM) | 5.5 | 20 | 20 (0.1 mg/ml) | 15 | 0 |
| Acetate (10 mM) | 5.5 | 20 | 80 (1 mg/ml) | 15 | 2 |
| Acetate (10 mM) | 5.5 | 0 | 80 (1 mg/ml) | 15 | 0 |

The exploratory stability study is designed for up to 24 months and selection was made based on 3 months data using the ranking method described in section 1.4. Samples were analyzed at different time points using the following analytical techniques: SEC, WCX, Light blockage, turbidity and DLS. The SEC three months data are shown in table 13. The final composition was decided based on three months data.

TABLE 13
Raw SEC data resulting from the exploratory stability for the four anti-
TNFAlpha antibody BS

| | | Results | | | | |
|---|---|---|---|---|---|---|
| | Storage | | % Main | | | Average |
| Composition¹ | temperatures | % HMW | Peak | % LMW | Ranking | Ranking |
| Mannitol 20 mg/ml, | −80° C. | 0.6 | 99.3 | 0.10 | 1 | 1.2 |
| Glycine 15 mg/ml, | −20° C. | 2.4 | 97.5 | 0.10 | 2 | |
| PS80 0.1% w/v | 5° C. | 0.6 | 99.3 | 0.10 | 1 | |
| | 25° C. | 0.8 | 98.2 | 0.90 | 1 | |
| | 40° C. | 2.3 | 94.2 | 3.40 | 1 | |
| Mannitol 20 mg/ml. | −80° C. | 0.6 | 99.3 | 0.10 | 1 | 1.2 |
| Glycine 15 mg/ml. | −20° C. | 2.5 | 97.4 | 0.10 | 2 | |
| PS20 0.01% w/v | 5° C. | 0.6 | 99.3 | 0.10 | 1 | |
| | 25° C. | 0.9 | 98.2 | 0.90 | 1 | |
| | 40° C. | 2.5 | 94.2 | 3.40 | 1 | |
| Mannitol 20 mg/ml, | −80° C. | 0.7 | 99.3 | 0.10 | 1 | 1 |
| Glycine 15 mg/ml, | −20° C. | 1.5 | 98.4 | 0.10 | 1 | |
| PS80 0.1% w/v, | 5° C. | 0.7 | 99.2 | 0.10 | 1 | |
| NaCl 2 mg/ml | 25° C. | 0.9 | 98.2 | 0.90 | 1 | |
| | 40° C. | 2.6 | 94.2 | 3.30 | 1 | |
| Glycine 15 mg/ml, | −80° C. | 0.7 | 99.3 | 0.10 | 1 | 2.2 |
| PS20 0.01% | −20° C. | 3.4 | 96.5 | 0.10 | 4 | |
| | 5° C. | 0.6 | 99.3 | 0.10 | 1 | |
| | 25° C. | 1 | 98.1 | 0.90 | 1 | |
| | 40° C. | 4.8 | 91.7 | 3.50 | 4 | |

¹All compositions are in 10 mM acetate buffer at pH 5.5
The final decision is based on the ranking resulting from all analytical technics (SEC, WCX, Light blockage, turbidity and DLS) that were used. The two best ranked compositions after 3 months were defined as the two promising candidates (see table 14).

TABLE 14
General ranking after 3 months stability for new developed compositions

| | Storage | | | | Average |
|---|---|---|---|---|---|
| Composition¹ | Temperature | Physical | Chemical | Ranking | Ranking |
| Mannitol 20 mg/ml, | −80° C. | 1.20 | 1 | 1.1 | 1.2 |
| Glycine 15 mg/ml and | −20° C. | 1.40 | 1 | 1.2 | |
| PS80 0.1% w/v | 5° C. | 1.20 | 1 | 1.1 | |
| | 25° C. | 1.20 | 1 | 1.1 | |
| | 40° C. | 1.00 | 2 | 1.5 | |
| Mannitol 20 mg/ml, Glycine | −80° C. | 1.20 | 1 | 1.1 | 1.2 |
| 15 mg/ml and PS20 0.01% w/v | −20° C. | 1.40 | 1 | 1.2 | |
| | 5° C. | 1.20 | 1 | 1.1 | |
| | 25° C. | 1.20 | 1 | 1.1 | |
| | 40° C. | 1.00 | 2 | 1.5 | |
| Mannitol 20 mg/ml, Glycine | −80° C. | 1.60 | 1 | 1.3 | 1.26 |
| 15 mg/ml, PS80 0.1% w/v and | −20° C. | 1.80 | 1 | 1.4 | |
| NaCl 2 mg/ml | 5° C. | 1.60 | 1 | 1.3 | |
| | 25° C. | 1.60 | 1 | 1.3 | |
| | 40° C. | 1.00 | 1 | 1 | |
| Glycine 15 mg/ml and | −80° C. | 1.20 | 1 | 1.1 | 1.65 |
| PS20 0.01% w/v | −20° C. | 3.40 | 1 | 2.2 | |
| | 5° C. | 1.00 | 1 | 1 | |
| | 25° C. | 2.40 | 1 | 1.7 | |
| | 40° C. | 2.50 | 2 | 2.25 | |

¹All compositions are in 10 mM acetate buffer at pH 5.5

### 2.2.2 Isothermal Stability Studies:

Short term accelerated stability studies were performed in order to support the selected composition based on the 3 months data (table 14). The SEC raw data obtained after exposing the compositions to isothermal stress of 1 week at 55° C. are shown in table 15 to exemplify one of the parameters used for ranking. The final ranking of this study, shown in table 16, shows the difference between the 4 compositions more clearly; accordingly, the composition having 20 mg/ml mannitol, 20 mg/ml, glycine 15 mg/ml and PS80 at 0.1% w/v is one of the best compositions under the tested conditions (table 16).

TABLE 15
SEC on 4 prototype compositions before and after
isothermal stress

| | Storage | Results | | | |
|---|---|---|---|---|---|
| | temperature | % | % Main | % | |
| Composition¹ | and time | HMW | Peak | LMW | Ranking |
| Mannitol | T0 | 0.30 | 99.63 | 0.08 | 1 |
| 20 mg/ml, | 55° C. for | 1.01 | 96.00 | 0.80 | 1 |
| Glycine 15 mg/ml, | 1 week | | | | |
| PS80 0.1% w/v | | | | | |
| Mannitol | T0 | 0.29 | 99.60 | 0.11 | 1 |
| 20 mg/ml, | 55° C. for | 1.07 | 96.27 | 0.77 | 1 |
| Glycine 15 mg/ml, | 1 week | | | | |
| PS20 0.01% w/v | | | | | |
| Mannitol | T0 | 0.34 | 99.58 | 0.08 | 1 |
| 20 mg/ml, | 55° C. for | 1.43 | 94.64 | 0.76 | 4 |
| Glycine 15 mg/ml, | 1 week | | | | |
| PS80 0.1% w/v, | | | | | |
| NaCl 2 mg/ml | | | | | |
| Glycine 15 mg/ml, | T0 | 0.33 | 99.61 | 0.06 | 1 |
| PS20 0.01% w/v | 55° C. for | 1.27 | 95.20 | 0.75 | 3 |
| | 1 week | | | | |

¹All compositions are in 10 mM acetate buffer at pH 5.5

TABLE 16
General ranking after isothermal stress

| | Temperature/ | | | Aver- | Overall |
|---|---|---|---|---|---|
| Composition¹ | Time | Physical | Chemical | age | Ranking |
| Mannitol 20 mg/ml, | 50° C./ | 1 | 1.33 | 1.165 | 1.0825 |
| | 1 month | | | | |
| Glycine 15 mg/ml, | 55° C./ | 1 | 1 | 1 | |
| PS80 0.1% w/v | 1 Week | | | | |
| Mannitol 20 mg/ml, | 50° C./ | 2 | 1.33 | 1.665 | 1.5825 |
| | 1 month | | | | |
| Glycine 15 mg/ml, | 55° C./ | 2 | 1 | 1.5 | |
| PS20 0.01% w/v | 1 Week | | | | |
| Mannitol 20 mg/ml, | 50° C./ | 4 | 1 | 2.5 | 2.5 |
| | 1 month | | | | |
| Glycine 15 mg/ml, | 55° C./ | 4 | 1 | 2.5 | |
| PS80 0.1% w/v, | 1 Week | | | | |
| NaCl 2 mg/ml | | | | | |
| Glycine 15 mg/ml, | 50° C./ | 3 | 1.33 | 2.165 | 2.0825 |
| | 1 month | | | | |
| PS20 0.01% w/v | 55° C./ | 3 | 1 | 2 | |
| | 1 Week | | | | |

¹All compositions are in 10 mM acetate buffer at pH 5.5

### 2.2.3 Accelerated Stability Studies

The 4 selected compositions as shown in table 16 were further subjected to additional accelerated stability studies together with two alternative compositions (histidine buffer (7.45 mM, pH 6), mannitol 12, PS80 0.1% w/v, NaCl 6.165 mg/ml and acetate buffer (10 mM, pH5.5), mannitol 12, PS80 0.1% w/v, NaCl 6.165 mg/ml). The 6 compositions were tested for mechanical stability and freeze/thaw stability and samples were analyzed before and after stress using the following analytical techniques: SEC, Light blockage, and DLS. The SEC raw data that were obtained after applying a mechanical stress of 200 rpm for 3 hrs are shown in table 17 to exemplify one of the parameters used for ranking.

TABLE 17
SEC raw data of 6 compositions prior and after mechanical stress
(200 rpm, 3 hrs)

| | Stress | Results | | | |
|---|---|---|---|---|---|
| | conditions: | | % Main | | |
| Composition | stress and time | % LMW | Peak | % HMW | Ranking |
| Acetate buffer (10 mM, pH 5.5), | At T0 | 0.08 | 99.63 | 0.30 | 1 |
| Mannitol 20 mg/ml, Glycine | After 3 hr at | 0.08 | 100.49 | 0.34 | 1 |
| 15 mg/ml, PS80 0.1% w/v | 200 rpm | | | | |
| Acetate buffer (10 mM, pH 5.5), | At T0 | 0.11 | 99.60 | 0.29 | 1 |
| Mannitol 20 mg/ml, Glycine | After 3 hr at | 0.10 | 100.37 | 0.32 | 1 |
| 15 mg/ml, PS20 0.01% w/v | 200 rpm | | | | |
| Acetate buffer (10 mM, pH 5.5), | At T0 | 0.08 | 99.58 | 0.34 | 1 |
| Mannitol 20 mg/ml, Glycine | After 3 hr at | 0.08 | 100.13 | 0.35 | 1 |
| 15 mg/ml, PS80 0.1% w/v, NaCl | 200 rpm | | | | |
| 2 mg/ml | | | | | |
| Acetate buffer (10 mM, pH 5.5), | At T0 | 0.06 | 99.61 | 0.33 | 1 |
| Glycine 15 mg/ml, PS20 0.01% w/v | After 3 hr at | 0.07 | 99.52 | 0.33 | 1 |
| | 200 rpm | | | | |
| Histidine buffer (7.45 mM pH 6), | At T0 | 0.08 | 99.37 | 0.56 | 1 |
| Mannitol 12, PS80 0.1% w/v, NaCl | After 3 hr at | 0.13 | 99.40 | 0.57 | 1 |
| 6.165 mg/ml | 200 rpm | | | | |
| Acetate buffer (10 mM, pH5.5), | At T0 | 0.07 | 99.38 | 0.55 | 1 |
| Mannitol 12, PS80 0.1% w/v, NaCl | After 3 hr at | 0.08 | 99.06 | 0.53 | 1 |
| 6.165 mg/ml | 200 rpm | | | | |

SEC raw data obtained after the 5th cycle of freeze/thaw stress are shown in table 18 to exemplify one of the parameters used for ranking.

TABLE 18
SEC raw data of 6 compositions prior and after Freeze/thaw stress

| | Stress | Results | | | |
|---|---|---|---|---|---|
| | conditions: | | % Main | | |
| Composition | stress and time | % HMW | Peak | % LMW | Ranking |
| Acetate buffer (10 mM, pH 5.5), | At T0 | 0.30 | 99.63 | 0.08 | 1 |
| Mannitol 20 mg/ml, Glycine | 5th cycle | 0.32 | 100.96 | 0.09 | 1 |
| 15 mg/ml, PS80 0.1% w/v | | | | | |
| Acetate buffer (10 mM, pH 5.5), | At T0 | 0.29 | 99.60 | 0.11 | 1 |
| Mannitol 20 mg/ml, Glycine | 5th cycle | 0.35 | 101.68 | 0.10 | 1 |
| 15 mg/ml, PS20 0.01% w/v | | | | | |
| Acetate buffer (10 mM, pH 5.5), | At T0 | 0.34 | 99.58 | 0.08 | 1 |
| Mannitol 20 mg/ml, Glycine | 5th cycle | 0.38 | 100.95 | 0.09 | 1 |
| 15 mg/ml, PS80 0.1% w/v, NaCl | | | | | |
| 2 mg/ml | | | | | |
| Acetate buffer (10 mM, pH 5.5), | At T0 | 0.33 | 99.61 | 0.06 | 1 |
| Glycine 15 mg/ml, PS20 0.01% w/v | 5th cycle | 0.43 | 100.16 | 0.08 | 1 |
| Histidine buffer (7.45 mM pH 6), | At T0 | 0.56 | 99.37 | 0.08 | 1 |
| Mannitol 12, PS80 0.1% w/v, NaCl | 5th cycle | 0.60 | 100.03 | 0.09 | 1 |
| 6.165 mg/ml | | | | | |
| Acetate buffer (10 mM, pH 5.5), | At T0 | 0.55 | 99.38 | 0.07 | 1 |
| Mannitol 12, PS80 0.1% w/v, NaCl | 5th cycle | 0.55 | 100.03 | 0.07 | 1 |
| 6.165 mg/ml | | | | | |

The final ranking obtained according to the mechanical stability and the freeze/thaw stability studies are shown in table 19 and 20.

TABLE 19
General ranking after mechanical stress

| | | Methods | | | | | |
|---|---|---|---|---|---|---|---|
| | | Rank | Rank | Rank | | | Overall |
| Composition | Stress | SEC | DLS | HIAC | Average | Rank | ranking |
| Acetate buffer (10 mM, pH | 100 rpm | 1.00 | 3 | 1 | 1.67 | 2 | 1.5 |
| 5.5), Mannitol 20 mg/ml, | 200 rpm | 1.00 | 2.33333 | 1 | 1.44 | 1 | |
| Glycine 15 mg/ml, PS80 | | | | | | | |
| 0.1% w/v | | | | | | | |
| Acetate buffer (10 mM, pH | 100 rpm | 1.00 | 4.66667 | 1 | 2.222222 | 5 | 4.5 |
| 5.5), Mannitol 20 mg/ml, | 200 rpm | 1.00 | 3.33333 | 1.5 | 1.944444 | 4 | |
| Glycine 15 mg/ml, PS20 | | | | | | | |
| 0.01% w/v | | | | | | | |
| Acetate buffer (10 mM, pH | 100 rpm | 1.00 | 3 | 1 | 1.666667 | 2 | 2.5 |
| 5.5), Mannitol 20 mg/ml, | 200 rpm | 1.00 | 3 | 1.5 | 1.833333 | 3 | |
| Glycine 15 mg/ml, PS80 | | | | | | | |
| 0.1% w/v, NaCl 2 mg/ml | | | | | | | |
| Acetate buffer (10 mM, pH | 100 rpm | 1.00 | 3 | 1.5 | 1.833333 | 4 | 3 |
| 5.5), Glycine 15 mg/ml, | 200 rpm | 1.00 | 2.33333 | 1.5 | 1.611111 | 2 | |
| PS20 0.01% w/v | | | | | | | |
| Histidine buffer (7.45 mM pH | 100 rpm | 4.33 | 1.66667 | 2 | 2.666556 | 6 | 6 |
| 6), Mannitol 12, PS80 | 200 rpm | 2.67 | 1.66667 | 2 | 2.111222 | 6 | |
| 0,1% w/v, NaCl 6.165 mg/ml | | | | | | | |
| Acetate buffer (10 mM, | 100 rpm | 1.00 | 1.66667 | 1.5 | 1.388889 | 1 | 2.5 |
| pH 5.5), Mannitol 12, PS80 | 200 rpm | 1.00 | 3.33333 | 1.5 | 1.944444 | 4 | |
| 0.1% w/v, NaCl 6.165 mg/ml | | | | | | | |

The mechanical stability showed a significant better stability for the composition comprising acetate buffer (10 mM, pH 5.5), mannitol 20 mg/ml, glycine 15 mg/ml, PS80 0.1% w/v over all other composition.

TABLE 20
General ranking after freeze/thaw stress

| | | Methods | | | | |
|---|---|---|---|---|---|---|
| | | Rank | Rank | Rank | | Overall |
| Composition | Stress | SEC | DLS | HIAC | Average | Ranking |
| Acetate buffer (10 mM, pH 5.5), | 5 cycles | 1.00 | 1.25 | 1 | 1.08 | 2 |
| Mannitol 20 mg/ml, Glycine | F/T | | | | | |
| 15 mg/ml, PS80 0.1% w/v | | | | | | |
| Acetate buffer (10 mM, pH 5.5), | 5 cycles | 1.00 | 1 | 1 | 1.00 | 1 |
| Mannitol 20 mg/ml, Glycine | F/T | | | | | |
| 15 mg/ml, PS20 0.01% w/v | | | | | | |
| Acetate buffer (10 mM, pH 5.5), | 5 cycles | 1.00 | 4 | 1 | 2.00 | 4 |
| Mannitol 20 mg/ml, Glycine | F/T | | | | | |
| 15 mg/ml, PS80 0.1% w/v, NaCl | | | | | | |
| 2 mg/ml | | | | | | |
| Acetate buffer (10 mM, pH 5.5), | 5 cycles | 1.00 | 1.75 | 1 | 1.25 | 3 |
| Glycine 15 mg/ml, PS20 0.01% w/v | F/T | | | | | |
| Histidine buffer (7.45 mM pH 6), | 5 cycles | 1.00 | 5 | 1.5 | 2.50 | 6 |
| Mannitol 12 mg/ml, PS80 0.1% w/v | F/T | | | | | |
| NaCl 6,165 mg/ml | | | | | | |
| Acetate buffer (10 mM, pH 5.5), | 5 cycles | 1.00 | 5 | 1 | 2.33 | 5 |
| Mannitol 12 mg/ml, PS80 0.1% w/v, | F/T | | | | | |
| NaCl 6,165 mg/ml | | | | | | |

Freeze/thaw studies at −80° C. showed better stability for the composition acetate buffer (10 mM, pH 5.5), mannitol 20 mg/ml, glycine 15 mg/ml, PS20 0.01% w/v followed by acetate buffer (10 mM, pH 5.5), mannitol 20 mg/ml, glycine 15 mg/ml, PS80 0.1% w/v. However, the difference obtained for the freeze/thaw stability study is non-significant in view of the obtained raw data (not shown).

In order to reach a final decision the 6 compositions where further compared after 3 months. The final overall stability ranking is presented here in table 21.

TABLE 21
Final ranking of the compositions after 3 months storage at different temperatures

| | Storage | | | | Average |
|---|---|---|---|---|---|
| Composition | Temperature | Physical | Chemical | Ranking | Ranking |
| Acetate buffer (10 mM pH 5.5), | −80° C. | 1.75 | 1 | 1.375 | 1.67 |
| Mannitol 20 mg/ml, Glycine | −20° C. | 2.75 | 1 | 1.875 | |
| 15 mg/ml, PS80 0.1% w/v | 5° C. | 1.75 | 1 | 1.375 | |
| | 25° C. | 1.75 | 1 | 1.375 | |
| | 40° C. | 1.67 | 3 | 2.3 | |
| Acetate buffer (10 mM, pH 5.5), | −80° C. | 1.75 | 1 | 1.375 | 1.67 |
| Mannitol 20 mg/ml, Glycine | −20° C. | 2.75 | 1 | 1.875 | |
| 15 mg/ml, PS20 0.01% w/v | 5° C. | 1.75 | 1 | 1.375 | |
| | 25° C. | 1.75 | 1 | 1.375 | |
| | 40° C. | 1.67 | 3 | 2.3 | |
| Acetate buffer (10 mM, pH 5.5), | −80° C. | 1.75 | 1 | 1.375 | 1.67 |
| Mannitol 20 mg/ml, Glycine | −20° C. | 2.75 | 1 | 1.875 | |
| 15 mg/ml, PS80 0.1% w/v, NaCl | 5° C. | 1.75 | 1 | 1.375 | |
| 2 mg/ml | 25° C. | 1.75 | 1 | 1.375 | |
| | 40° C. | 1.67 | 3 | 2.3 | |
| Acetate buffer (10 mM. pH 5.5), | −80° C. | 1.75 | 1 | 1.375 | 2.25 |
| Glycine 15 mg/ml, PS20 | −20° C. | 5.25 | 1 | 3.125 | |
| 0.01% w/v | 5° C. | 1.75 | 1 | 1.375 | |
| | 25° C. | 1.75 | 1 | 1.375 | |
| | 40° C. | 5 | 3 | 4 | |
| Acetate buffer (10 mM, pH 5.5), | −80° C. | 2 | 1 | 1.5 | 1.825 |
| Mannitol 12 mg/ml, PS80 | −20° C. | 2.25 | 1 | 1.625 | |
| 0.1% w/v, NaCl 6.165 mg/ml | 5° C. | 2 | 1 | 1.5 | |
| | 25° C. | 4 | 1 | 2.5 | |
| | 40° C. | 3 | 1 | 2 | |
| Histidine buffer (7.45 mM pH 6), | −80° C. | 2 | 1 | 1.5 | 1.8 |
| Mannitol 12 mg/ml, PS80 0.1% w/v | −20° C. | 2 | 1 | 1.5 | |
| NaCl 6.165 mg/ml | 5° C. | 2 | 1 | 1.5 | |
| | 25° C. | 4 | 1 | 2.5 | |
| | 40° C. | 3 | 1 | 2 | |

Accordingly, one final composition (FC) as defined in table 22 was selected for further comparative studies with the market formulation.

TABLE 22
Composition of anti-TNFAlpha antibody BS composition
for phase 3

| | Composition | | Reference to |
|---|---|---|---|
| Components | mg/ml | Function | standards |
| anti-TNFAlpha antibody | 50.000 | Active substance | In-house |
| BS | | | |
| Sodium acetate tri | 1.170 | Buffering agent | USP |
| hydrate | | | |
| Acetic acid¹ | 0.080 | Buffering agent | USP |
| Mannitol | 20.000 | Tonicity agent | |
| Glycine | 15.000 | Buffering agent | USP |
| Polysorbate 80 | 1.000 | Surfactant, | NF |
| | | protein stabilizer | |
| Water for injection² | ad. 1 mL | Diluent | USP |

¹Acetic acid was used for pH adjustment (if needed) after adding all the excipients
²Water for injection manufactured by distillation according to Ph. Eur. is routinely tested to USP

### 3 Comparison of the Anti-TNFAlpha Antibody BS Composition for Phase III and the Originator Composition (OC) (Example 6)

### 3.1 Isothermal Accelerated Stability Study

The final composition (FC) as defined in table 22 and the originator composition (OC) were subjected to 55° C. for one week. The data of the final composition obtained from isothermal accelerated stability studies as described in 1.3 were used for a comparison with a similar study performed on the originator composition which is manufactured in the originator composition. In this case only SEC and WCX data were available for comparison. The results of this comparative analysis are presented in table 23.

TABLE 23
Comparison between the selected composition and the
market composition with regards to stability at 55° C.
for 1 week

| | | SEC | | |
|---|---|---|---|---|
| Composition | Time point | % LMW | % Monomer | % HMW |
| FC | T zero | 0.08 | 99.63 | 0.30 |
| | 1 week | 0.81 | 98.15 | 1.03 |
| OC | T zero | 0.10 | 99.30 | 0.60 |
| | 1 week | 4.40 | 88.30 | 7.40 |
| | | WCX | | |
| FC | T zero | 17 | 65 | 18 |
| | 1 week | 48 | 34 | 17 |
| OC | T zero | 16 | 63 | 21 |
| | 1 week | 46 | 29 | 25 |

The results show a significant better stability for the selected final composition (FC) in comparison to the market composition (OC).

Furthermore, the comparison of data shown in tables 23 and 15 indicate that the other compositions of the invention assayed as reported in table 1 also provided an improved stability for the antibody anti-TNFAlpha antibody BS in comparison to the market formulation. Therefore all 6 compositions are capable of increasing thermal stability in comparison to the market formulation because for all 6 compositions the amount of active monomer is more than 94% in comparison to the total peak area whereas the market formulation contains only an amount of monomer of about 88%.

### 3.2 Mechanical Stability

In this study the mechanical stability of the final composition (FC) was compared with the mechanical stability of the market composition (OC) by using the anti-TNF Alpha antibody BS, wherein the final composition was stirred at 200 rpm stirring for 3 hrs at 200 rpm and the market composition for 2 hrs. For evaluation: SEC, DLS and Light blockage (HIAC) were used (table 24).

No difference was seen with regards to soluble aggregates. The differences were mainly in the sub-visible particle formation and smaller aggregates as seen by both light blockage and dynamic light scattering (DLS). Although the final composition (FC) was more stressed than the marketed composition (OC) (3 hours vs. 2 hours) it showed higher mechanical stability especially with regards to particle formation.

TABLE 24
Comparison between the selected composition (FC) and the market
composition (OC) with regards to mechanical stability

| | | Light Blockage (HIAC) | | |
|---|---|---|---|---|
| Composition | Time point | >1 μm | >10 μm | >25 μm |
| FC | T zero | 280 | 18 | 3 |
| | 3 hours | 5575 | 75 | 5 |
| OC | T zero | 1846 | 62 | 2 |
| | 2 hours | 54868 | 113 | 3 |

| | | DLS | | | |
|---|---|---|---|---|---|
| | | % monomer | % monomer | | |
| | | by Volume | by Intensity | Z-average | PDI |
| FC | T zero | 100 | 100 | 1.72 | 0.07 |
| | 3 hours | 100 | 36.5 | 8.74 | 0.89 |
| OC | T zero | 100 | 100 | 13.28 | 0.06 |
| | 2 hours | 98.7 | 48.1 | 51.80 | 0.54 |

### 4. Conclusion

The inventors used the anti-TNFAlpha antibody BS as an example molecule to develop a composition that improves antibody stability. The inventors initially wanted to stabilize the antibody against mechanical stress, however by using different screening methods and comparing different buffers, excipients such as polyols, sugars, amino acids, the presence of salt and different detergents the inventors achieved to develop compositions that not only protect the antibody against mechanical stress but also increase the stability to thermal stress.

Therefore, based on the anti-TNFAlpha antibody BS the inventors achieved to develop compositions that improve the mechanical stability as well as the thermal stability of the antibody in comparison to the reference market formulation. The different compositions showed very good results at many different stress conditions (e.g. high temperatures and sheer rates) and are very suitable for the desired storage temperature (2-8° C.). The developed compositions might as well improve the stability of other antibodies since the tested antibody anti-TNFAlpha antibody BS is an IgG antibody and thus shares high similarity with the amino acid composition of other IgG antibodies.

Furthermore, the developed compositions all improve mechanical stability as well as thermal stability of anti-TNFAlpha antibody BS in comparison to the composition that is on the market. The compositions contain more than 94% of the antibody in form of a functional monomer after one week at 55° C., the final selected composition contains even 98.15%, whereas the originator composition contains only 88.30% functional monomer. Increasing the functional monomer by 10% is a considerable gain for a molecule that is expensive in its production.

### 5. Study of the Stability of an Anti-CXCR5 Antibody in the Form of the Composition of the Invention (Example 7)

The composition comprising an anti-CXCR5 antibody, as defined in table 25 below was subjected to 5° C. or 40° C. for 1 to 6 months.

TABLE 25
Composition of anti-CXCR5 antibody used for stability tests

| | Composition | | Reference to |
|---|---|---|---|
| Components | mg/ml | Function | standards |
| anti-CXCR5 antibody | 41.000 | Active | In-house |
| SAR113244 | | substance | |
| Sodium acetate tri | 1.170 | Buffering agent | USP |
| hydrate | | | |
| Acetic acid¹ | 0.080 | Buffering agent | USP |
| Mannitol | 20.000 | Tonicity agent | |
| Glycine | 15.000 | Buffering agent | USP |
| Polysorbate 80 | 1.000 | Surfactant, | NF |
| | | protein stabilizer | |
| Water for injection² | ad. 1 mL | Diluent | USP |

¹Acetic acid was used for pH adjustment (if needed) after adding all the excipients
²Water for injection manufactured by distillation according to Ph. Eur. is routinely tested to USP

In the study, the long isothermal stability of the composition was assayed by storing the composition at 40° C. for 1 month, 3 months and 6 months and comparing it to the same composition stored at 5° C. for the same periods. For evaluation, DLS (table 26) and Light blockage (HIAC, table 27) were used.

The results clearly show that the anti-CXCR5 antibody is stable over a prolonged period of time, even under accelerated conditions, with the formulation of the invention.

TABLE 26
Measurement of protein aggregation of the anti-CXCR5 antibody
composition determined by DLS

| | | DLS | | | |
|---|---|---|---|---|---|
| Time | Temperature | % monomer | % monomer | | |
| point | of storage | by Volume | by Intensity | Z-average | PDI |
| T zero | — | 100 | 100 | 5.03 | 0.076 |
| 3 months | +5° C. | 100 | 100 | 4.978 | 0.053 |
| 3 months | +40° C. | 100 | 100 | 6.41 | 0.113 |

TABLE 27
Subvisible particles of the anti-CXCR5 antibody
composition determined by Light blockage

| Light Blockage (HIAC) | | |
|---|---|---|
| Time point | | |
| T zero | 1 month | 6 month |

| | Temperature | | | | |
|---|---|---|---|---|---|
| | — | +5° C. | +40° C. | +5° C. | +40° C. |
| 1.5 μm | 530 | 120 | 197 | 780 | 200 |
| 2.0 μm | 328 | 62 | 128 | 307 | 118 |
| 5.0 μm | 95 | 18 | 38 | 73 | 40 |
| 10.0 μm | 20 | 5 | 13 | 20 | 18 |
| 15.0 μm | 7 | 2 | 5 | 10 | 8 |
| 25.0 μm | 0 | 0 | 0 | 3 | 5 |

These results thus confirm that the formulation of the invention enables increasing stability of an monoclonal antibody.

1. A pharmaceutical composition comprising:
a) an antibody,
b) at least one buffer agent selected from the group consisting of acetate and histidine,
c) at least one amino acid selected from the group consisting of glycine, asparagine and glutamine, and/or at least one excipient selected from the group consisting of threhalose and mannitol, and
d) a surfactant,
wherein the pH of the composition is 5.0 to 6.5.

2. The composition according to claim 1, wherein the antibody is an IgG antibody.

3. The composition according to claim 3, wherein the composition comprises 5 to 15 mM of at least one buffer agent that is optionally acetate or histidine.

4-5. (canceled)

6. The composition according to claim 1, wherein the composition comprises 1 to 70 mg/ml of at least one excipient, optionally wherein the at least one excipient is mannitol.

7. (canceled)

8. The composition according to claim 1, wherein the composition comprises less than 7 mg/ml sodium chloride.

9. The composition according to claim 1, wherein:
the surfactant is polysorbate;
the composition comprises 0.001% w/v to 0.15% w/v surfactant; or
the composition comprises 1 mg/ml to 30 mg/ml of at least one amino acid that is optionally glycine.

10-12. (canceled)

13. The composition according to claim 1, comprising
a) 40 to 50 mg/ml antibody,
b) 5 to 15 mM acetate buffer or histidine buffer,
c) 20 mg/ml mannitol, and/or 15 mg/ml glycine, and
d) 0.1% w/v polysorbate 80 or 0.01% w/v polysorbate 20,
wherein the pH is 5.0 to 6.5.

14. The composition according to claim 1, comprising:
a) 50 mg/ml antibody, and
b) 5 to 15 mM acetate buffer or histidine buffer, and
c) 20 mg/ml mannitol and/or 15 mg/ml glycine, and
d) 0.1% w/v polysorbate 80 or 0.01% w/v polysorbate 20,
wherein the pH is 5.0 to 6.5.

15. The composition according to claim 1, comprising
a) 50 mg/ml antibody, and
b) 10 mM acetate buffer, and
c) 20 mg/ml mannitol, and 15 mg/ml glycine, and
d) 0.1% w/v polysorbate 80,
wherein the pH is 5.5.

16. The composition according to claim 15, comprising
a) 50 mg/ml antibody,
b) 1.17 mg/ml sodium acetate trihydrate and 0.08 mg/ml acetic acid,
c) 20 mg/ml mannitol, and 15 mg/ml glycine, and
d) 0.1% w/v polysorbate 80,
wherein the pH is 5.5.

17. The composition according to claim 1, wherein:
the composition is for intravenous administration, intramuscular, intraperitoneal, intracerobrospinal, subcutaneous, intra-articular, intrasynovial or intrathecal administration;
the antibody comprises a heavy chain consisting of the amino acid sequence represented by SEQ ID NO: 1 and a light chain consisting of the amino acid sequence represented by SEQ ID NO: 2; or
the antibody is Adalimumab.

18. The composition according to claim 1, wherein the composition has at least one feature selected from the group consisting of:
(a) decreased amount of aggregates after storage at about 55° C. for one week as measured by Size Exclusion Chromatography (SEC),
(b) higher amount of monomers after storage at about 55° C. for one week as measured by SEC, and
(c) less fragments after storage at about 55° C. for one week as measured by SEC, compared to a reference composition.

19. The composition according to claim 1, wherein the composition has at least one feature selected from the group consisting of:
(a) the composition is stable to thermal stress of 1 week at 55° C.,
(b) the composition is stable to mechanical stress of stirring for 3 hours at 55° C., and/or
(c) the composition is stable to stress resulting from freezing and thawing, wherein freezing and thawing refers to freezing the composition at −80° C. for 24 hrs followed by thawing at room temperature for 90 min, wherein the cycle is repeated for 5 times repeated and in the 5th cycle the temperature is kept for 72 hrs at −80° C.

20. The composition according to claim 19, wherein stable refers to at least one of the following characteristics
i) the composition has a monomer content in % of more than 90% in relation to the total area of all peaks when measured by SEC,
ii) the composition has an aggregate content in % of less than 3% in relation to the total area of all peaks when measured by SEC, and/or
iii) the composition has a fragment content in % of less than 3% in relation to the total area of all peaks when measured by SEC.

21. The composition according to claim 1, wherein the composition has at least one feature selected from the group consisting of:
(a) decreased amount of aggregates after storage at about 40° C. for 1 to 6 months as measured by Light blockage/Light obscuration (LO), and
(b) higher amount of monomers after storage at about 40° C. for three months as measured by Dynamic Light Scattering (DLS).

22. The composition according to claim 1, wherein the composition is stable to thermal stress of 1 to 6 months at 40° C.

23. The composition according to claim 22, wherein stable refers to at least one of the following characteristics
i) the composition has a monomer content in % of more than 90% when analyzed by volume measured by DLS,
ii) the composition has a monomer content in % of more than 90% when analyzed by intensity measured by DLS, and/or
iii) the composition has less than 6000 particles >10 μm, less than 600 particles >25 μm and less than 10000 particles >1 μm when measured by LO.

24-27. (canceled)

28. A method of treating or preventing a disease or disorder comprising administering a subject in need thereof a therapeutically effective amount of the pharmaceutical composition of claim 1.

29. A kit comprising at least one container comprising the pharmaceutical composition according to claim 1 and an injection device.

30. A method for reducing aggregation and/or fragmentation of a therapeutic monoclonal antibody using the composition according to claim 1.
